(19) United States
(12) Patent Application Publication
(10) Pub. No.: US 2026/0035359 A1
(43) Pub. Date: Feb. 5, 2026

(54) HERBICIDAL IMIDAZOLE COMPOUNDS

(71) Applicant: SYNGENTA CROP PROTECTION AG, Basel (CH)

(72) Inventors: Mary Bernadette ASPINALL, Bracknell, Berkshire (GB); Emma BRIGGS, Bracknell, Berkshire (GB); Suzanna Jane DALE, Bracknell, Berkshire (GB); Philip Michael ELVES, Bracknell, Berkshire (GB); Charles William Frederick KINGSTON, Bracknell, Berkshire (GB); James Alan MORRIS, Bracknell, Berkshire (GB); Vikas SIKERVAR, Bracknell, Berkshire (GB); Samuel Vaughan WATKIN, Bracknell, Berkshire (GB)

(73) Assignee: SYNGENTA CROP PROTECTION AG, Basel (CH)

(21) Appl. No.: 18/995,007

(22) PCT Filed: Jul. 7, 2023
(86) PCT No.: PCT/EP2023/068870

§ 371 Date: Jan. 15, 2025

(30) Foreign Application Priority Data
Jul. 15, 2022 (GB) 2210443.4

(51) Int. Cl. C07D 403/06; A01N 43/90; A01P 13/00; A01P 19/00; C07D 403/14; C07D 405/06

(52) U.S. Cl. CPC C07D 403/06; A01N 43/90; A01P 13/00; A01P 19/00; C07D 403/14; C07D 405/06

(57) ABSTRACT

The present invention relates to compounds of Formula (I), or an agro-nomically acceptable salt of said compounds wherein A, Q, R¹, R², R³ and m are as defined herein. The invention further relates to herbicidal compositions which comprise a compound of Formula (I) and to the use of compounds of Formula (I) for controlling weeds, in particular in crops of useful plants.

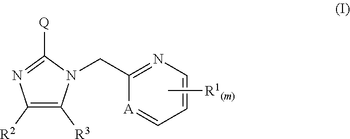

The present invention relates to herbicidal compounds, to processes for their preparation, to herbicidal compositions which comprise the compounds, and to their use for controlling weeds, in particular in crops of useful plants, or for inhibiting plant growth.

Certain imidazole compounds are disclosed in the prior art, for example in CN 106317072 and WO2022/015975. However, the potential herbicidal use of such imidazole compounds has never been reported.

Thus, according to the present invention there is provided a compound of Formula (I):

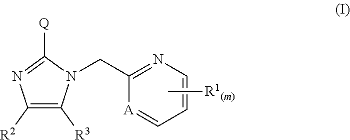

or an agronomically acceptable salt thereof,
wherein
A is CR¹¹ or N;
Q is selected from the group consisting of C₂-C₈ alkyl, C₂-C₈ alkenyl, C₂-C₈ alkynyl, C₁-C₈ haloalkyl, C₂-C₈ haloalkenyl, C₂-C₈ haloalkynyl, C₁-C₄alkoxy-C₁-C₈alkyl-, C₁-C₄haloalkoxy-C₁-C₈alkyl-, C₁-C₄alkoxy-C₁-C₈haloalkyl-, C₁-C₈cyanoalkyl- (e.g —CH₂CH₂CN), C₃-C₆ cycloalkyl (optionally substituted by CN, fluoro or chloro), —S(O)ₚR⁴ and —(CH₂)ₙR¹⁰; R¹ is independently selected from the group consisting of halogen, —CN, C₁-C₂alkyl, C₁-C₂haloalkyl, C₃-C₆cycloalkyl, C₁-C₂alkoxy- and C₁-C₂haloalkoxy-; R² is selected from the group consisting of halogen, —CN, NO₂, C₁-C₄alkyl, C-C₄haloalkyl, C₁-C₄alkoxy, —C(O)C₁-C₄alkyl, —C(O)OC₁-C₄alkyl, C1-C₄haloalkoxy, —S(O)pC₁-C₄alkyl, —C(R⁶)═NOR⁷ and C₃-C₆cycloalkyl;
R³ is selected from the group consisting of hydrogen, halogen, C₁-C₄ alkyl, C₁-C₄ haloalkyl, C₁-C₄ alkoxy, C₁-C₄ haloalkoxy, —CN, NO₂, C₂-C₄alkenyl, C₂-C₄alkynyl, —S(O)ₚC₁-C₄alkyl, —S(O)ₚC₁-C₄haloalkyl, —C(O)OC₁-C₄alkyl and —C(O)NR⁸R⁹;
R⁴ is selected from the group consisting of CrC₄alkyl, C₁-C₄haloalkyl and C₃-C₆cycloalkyl;
R⁵ is selected from the group consisting of hydrogen, methyl, ethyl, —(CH₂)—cyclopropyl, C₂-C₃ alkenyl, C₂-C₃ alkynyl and cyclopropyl;
R⁶ is selected from the group consisting of hydrogen, methyl and ethyl;
R⁷ is methyl or ethyl;
R⁸ is hydrogen or C₁-C₄alkyl;
R⁹ is hydrogen or C₁-C₄alkyl;
R¹⁰ is selected from the group consisting of —OH, —C(H)O, —C(O)R⁴, —S(O)ₚR⁴, —C(H)(OH)—CH₂OH, —C(R⁶)═NOR⁵, —NR¹²R¹³, C₃-C₆ cycloalkyl (optionally substituted by CN, fluoro or hydroxy), a C₃-C₆ saturated heterocycle comprising one or more oxygen atoms (e.g tetrahydropyran and tetrahydrofuran), and a 5-membered heteroaryl (e.g pyrazolyl) which comprises from 1 to 3 heteroatoms each independently selected from the group consisting of oxygen, nitrogen and sulphur, wherein said heteroaryl is optionally substituted by one or two substituents independently selected from the group consisting of halogen, cyano, C₁C₂ alkyl, C₁-C₂ haloalkyl, C₃-C₄cycloalkyl, C₁-C₂ alkoxy and C₁-C₄ haloalkoxy; R¹¹ is selected from the group consisting of hydrogen, fluoro, chloro and CN; R¹² and R¹³ are independently selected from the group consisting of C₁-C₄alkyl, C₃-C₄cycloalkyl, C—C₄alkyl-S(O)—, C₃-C₄cycloalkyl-S(O)p—, C₁-C₄alkyl-C(O)—and C₃-C₄cycloalkyl-C(O)—;
m=1 or 2;
n=1, 2 or 3; and p=0, 1 or 2.
C₁-C₄alkyl- and C₁-C₆alkyl- includes, for example, methyl (Me, CH₃), ethyl (Et, C₂H₅), n-propyl (n-Pr), isopropyl (i-Pr), n-butyl (n-Bu), isobutyl (i-Bu), sec-butyl and tert-butyl (t-Bu). C₁-C₂alkyl is methyl (Me, CH₃) or ethyl (Et, C₂H₅).

Halogen (or halo) includes, for example, fluorine, chlorine, bromine or iodine.

The same correspondingly applies to halogen in the context of other definitions, such as haloalkyl.

C₁-C₈haloalkyl- includes, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoropropyl and 2,2,2-trichloroethyl, heptafluoro-n-propyl and perfluoro-n-hexyl.

C₁-C₄haloalkyl- and C₁-C₂haloalkyl include, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, or 1,1-difluoro-2,2,2-trichloroethyl.

C₁-C₄alkoxy and C₁-C₈alkoxy includes, for example, methoxy and ethoxy.

C₁-C₈ haloalkoxy- and C₁-C₈ haloalkoxy- include, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy or 2,2,2-trichloroethoxy, preferably difluoromethoxy, 2-chloroethoxy or trifluoromethoxy.

C₂-C₈alkenyl- includes, for example, —CH═CH₂ (vinyl) and —CH₂—CH═CH₂ (allyl).

C₂-C₈alkynyl- refers to a straight or branched hydrocarbon chain radical group consisting solely of carbon and hydrogen atoms, containing at least one triple bond, having from two to four carbon atoms, and which is attached to the rest of the molecule by a single bond. Examples of C₂-C₄alkynyl include, but are not limited to, prop-1-ynyl, propargyl (prop-2-ynyl), and but-1-ynyl.

C₁-C₄alkyl-S— (alkylthio) includes, for example, methylthio, ethylthio, propylthio, isopropylthio, n-butylthio, isobutylthio, sec-butylthio or tert-butylthio, preferably methylthio or ethylthio.

C₁-C₄alkyl-S(O)— (alkylsulfinyl) includes, for example, methylsulfinyl, ethylsulfinyl, propylsulfinyl, isopropylsulfinyl, n-butylsulfinyl, isobutylsulfinyl, sec-butylsulfinyl or tert-butylsulfinyl, preferably methylsulfinyl or ethylsulfinyl.

C₁-C₄alkyl-S(O)₂— (alkylsulfonyl) includes, for example, methylsulfonyl, ethylsulfonyl, propylsulfonyl, isopropylsulfonyl, n-butylsulfonyl, isobutylsulfonyl, sec-butylsulfonyl or tert-butylsulfonyl, preferably methylsulfonyl or ethylsulfonyl.

In one embodiment of the present invention, A is N. In another embodiment of the present invention m is 1. Thus, in a preferred embodiment of the present invention there is provided a compound of Formula (Ia)

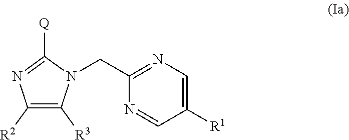

wherein Q, R¹, R² and R³are as defined above.

In one embodiment of the present invention, A is CR¹¹ and R¹¹ is selected from the group consisting of hydrogen, fluoro, chloro and CN, preferably fluoro or hydrogen. Thus, in a preferred embodiment of the present invention there is provided a compound of Formula (Ib)

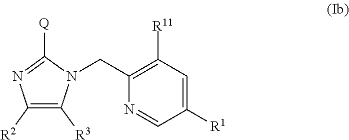

wherein Q, R¹, R², R³ and R¹¹ are as defined above.

In one embodiment of the present invention there is provided a compound of Formula (I), Formula (Ia) or Formula (Ib), wherein R¹ is chloro.

In one embodiment of the present invention there is provided a compound of Formula (I),Formula (Ia) or Formula (Ib), wherein R² is halogen (e.g C₁ or Br) or C-C₄haloalkyl (e.g —CF₃, —CHF₂).

In one embodiment of the present invention there is provided a compound of Formula (I), Formula (Ia), or Formula (Ib) wherein, R³ is hydrogen or halogen (e.g CI).

In a preferred embodiment of the present invention there is provided a compound of Formula (I), Formula (Ia), or Formula (Ib) wherein Q is selected from the group consisting of C₂-C₈ alkyl or C₂-C₆ alkyl (e.g —CH₂CH(CH₃)₂, n-butyl, n-pentyl), C₂-C₈ alkenyl (e.g —CH₂CH₂CH═CH₂), C₁-C₈ haloalkyl or C—C₆ haloalkyl (e.g —CH₂CH₂CHF₂, —CH₂CH₂CF₃, —CH₂CH₂CH₂CF₃), C₁-C₄alkoxy-C—C₈alkyl- (e.g —CH₂OCH₃, —CH₂CH₂OCH₃), C—C₄haloalkoxy-C₁-C₈alkyl- (e.g —CH₂OCH₂CHF₂, —CH₂CH₂OCH₂CHF₂), —S(O)ₚR⁴(e.g —SO₂CH₂CH₂CF₃, —S—CH₂CH₂CF₃) and —(CH₂)nR¹⁰ (e.g —CH₂CH₂CH₂OH, —CH₂CH₂C(H)(OH)CH₂OH, —CH₂CH₂C(H)O)—OH, —CH₂CH₂C(H)═N—OH, —CH₂CH₂C(H)═N—O—CH₃ and —CH₂CH₂C(H)═N—O—CH₂-cPr).

In one embodiment of the present invention there is provided a compound of Formula (I), Formula (Ia), or Formula (Ib) wherein Q is —(CH₂)nR¹⁰, n is 1, 2 or 3 and R¹⁰ is selected from the group consisting of —OH (e.g —CH₂CH₂CH₂OH), —C(H)(OH)—CH₂OH (e.g —CH₂CH₂C(H)(OH)CH₂OH), —C(H)═NOR⁵ (e.g —CH₂CH₂C(H)═N—OH, —CH₂CH₂C(H)═N—O—CH₃, —CH₂CH₂C(H)═N—O—CH₂-cPr), and —NR¹²R¹³ (e.g —CH₂CH₂CH₂N(CH₃)SO₂CH₃.

In a preferred embodiment of the present invention, there is provided a compound of Formula (I), Formula (Ia), or Formula (Ib) wherein Q is C₂-C₈ alkyl (e.g —CH₂CH(CH₃)₂, n-butyl, n-pentyl).

In another preferred embodiment of the present invention, there is provided a compound of Formula (I), Formula (Ia), or Formula (Ib) wherein Q is C₁-C₈ haloalkyl (e.g —CH₂CH₂CHF₂, —CH₂CH₂CF₃, —CH₂CH₂CH₂CF₃).

In another preferred embodiment of the present invention, there is provided a compound of Formula (I) or Formula (Ia), wherein Q is C₁-C₄haloalkoxy-C₁-C₈alkyl-e.g —CH₂OCH₂CHF₂, —CH₂CH₂OCH₂CHF₂).

The present invention further relates to compounds of Formula Int-1:

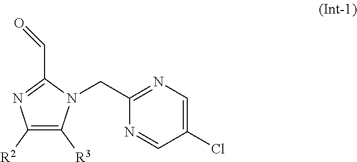

wherein R² and R³ are as defined with regard to Formula (I) above. In a preferred embodiment, R² is C₁-C₄haloalkyl (e.g —CHF₂ or —CF₃) and R³ is hydrogen.

In a more preferred embodiment the compound of Formula (Int-1) is compound 118.

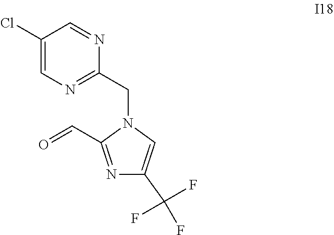 [SMILES: O=Cc1nc(C(F)(F)F)cn1Cc1ncc(Cl)cn1]

The present invention further relates to compounds of Formula (Illa-1):

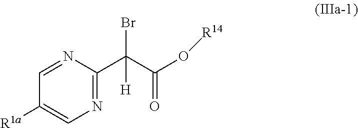

wherein R^{1a} is halogen (e.g F or C₁) and R¹⁴ is C₁-C₄ alkyl (e.g ethyl). In a more preferred embodiment the compound of Formula (Illa-1) is compound 128.

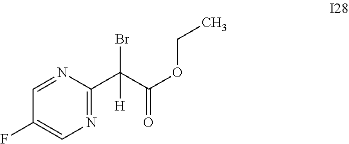

Compounds of Formula (I) may contain asymmetric centres and may be present as a single enantiomer, pairs of enantiomers in any proportion or, where more than one asymmetric centre are present, contain diastereoisomers in all possible ratios. Typically one of the enantiomers has enhanced biological activity compared to the other possibilities.

The present invention also provides agronomically acceptable salts of compounds of Formula (I). Salts that the compounds of Formula (I) may form with amines, including primary, secondary and tertiary amines (for example ammonia, dimethylamine and triethylamine), alkali metal and alkaline earth metal bases, transition metals or quaternary ammonium bases are preferred.

The compounds of Formula (I) according to the invention can be used as herbicides by themselves, but they are generally formulated into herbicidal compositions using formulation adjuvants, such as carriers, solvents and surface-active agents (SAA). Thus, the present invention further provides a herbicidal composition comprising a herbicidal compound according to any one of the previous claims and an agriculturally acceptable formulation adjuvant. The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil or solvents.

The herbicidal compositions generally comprise from 0.1 to 99% by weight, especially from 0.1 to 95% by weight, compounds of Formula I and from 1 to 99.9% by weight of a formulation adjuvant which preferably includes from 0 to 25% by weight of a surface-active substance.

The compositions can be chosen from a number of formulation types. These include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a soluble powder (SP), a wettable powder (WP) and a soluble granule (SG). The formulation type chosen in any instance will depend upon the particular purpose envisaged and the physical, chemical and biological properties of the compound of Formula (I).

Soluble powders (SP) may be prepared by mixing a compound of Formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility.

The mixture is then ground to a fine powder. Similar compositions may also be granulated to form water soluble granules (SG).

Wettable powders (WP) may be prepared by mixing a compound of Formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of Formula (I) and one or more powdered solid diluents or carriers, or from pre-formed blank granules by absorbing a compound of Formula (I) (or a solution thereof, in a suitable agent) in a porous granular material (such as pumice, attapulgite clays, fuller's earth, kieselguhr, diatomaceous earths or ground corn cobs) or by adsorbing a compound of Formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of Formula (I) in water or an organic solvent, such as a ketone, alcohol or glycol ether.

These solutions may contain a surface-active agent (for example to improve water dilution or prevent crystallisation in a spray tank).

Emulsifiable concentrates (EC) or oil-in-water emulsions (EW) may be prepared by dissolving a compound of Formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₃—C,a fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of Formula (I) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SAAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SAAs, to produce spontaneously a thermodynamically stable isotropic liquid formulation. A compound of Formula (I) is present initially in either the water or the solvent/SAA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of Formula (I).

SCs may be prepared by ball or bead milling the solid compound of Formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of Formula (I) may be dry milled and added to water, containing agents hereinbefore described, to produce the desired end product.

Aerosol formulations comprise a compound of Formula (I) and a suitable propellant (for example n-butane). A compound of Formula (I) may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of Formula (I) and, optionally, a carrier or diluent therefor. The polymeric shell may be produced by either an interfacial polycondensation reaction or by a coacervation procedure. The compositions may provide for controlled release of the compound of Formula (I) and they may be used for seed treatment. A compound of Formula (I) may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of Formula (I). Such additives include surface active agents (SAAs), spray additives based on oils, for example certain mineral oils or natural plant oils (such as soy bean and rape seed oil), modified plant oils such as methylated rape seed oil (MRSO), and blends of these with other bio-enhancing adjuvants (ingredients which may aid or modify the action of a compound of Formula (I).

Wetting agents, dispersing agents and emulsifying agents may be SAAs of the cationic, anionic, amphoteric or non-ionic type.

Suitable SAAs of the cationic type include quaternary ammonium compounds (for example cetyltrimethyl ammonium bromide), imidazolines and amine salts.

Suitable anionic SAAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butyinaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates, lignosulphonates and phosphates/sulphates of tristyrylphenols.

Suitable SAAs of the amphoteric type include betaines, propionates and glycinates.

Suitable SAAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); lecithins and sorbitans and esters thereof, alkyl polyglycosides and tristyrylphenols.

Suitable suspending agents include hydrophilic colloids (such as polysaccharides, polyvinylpyrrolidone or sodium carboxymethylcellulose) and swelling clays (such as bentonite or attapulgite).

The compounds of present invention can also be used in mixture with one or more additional herbicides and/or plant growth regulators. Examples of such additional herbicides or plant growth regulators include acetochlor, acifluorfen (including acifluorfen-sodium), aclonifen, ametryn, amicarbazone, aminopyralid, aminotriazole, atrazine, beflubutamid-M, benquitrione, bensulfuron (including bensulfuron-methyl), bentazone, bicyclopyrone, bilanafos, bipyrazone, bispyribac-sodium, bixlozone, bromacil, bromoxynil, butachlor, butafenacil, carfentrazone (including carfentrazone-ethyl), cloransulam (including cloransulam-methyl), chlorimuron (including chlorimuron-ethyl), chlorotoluron, chlorsulfuron, cinmethylin, clacyfos, clethodim, clodinafop (including clodinafop-propargyl), clomazone, clopyralid, cyclopyranil, cyclopyrimorate, cyclosulfamuron, cyhalofop (including cyhalofop-butyl), 2,4-D (including the choline salt and 2-ethylhexyl ester thereof), 2,4-DB, desmedipham, dicamba (including the aluminium, aminopropyl, bis-aminopropylmethyl, choline, dichloroprop, diglycolamine, dimethylamine, dimethylammonium, potassium and sodium salts thereof) diclosulam, diflufenican, diflufenzopyr, dimethachlor, dimethenamid-P, dioxopyritrione, diquat dibromide, diuron, epyrifenacil, ethalfluralin, ethofumesate, fenoxaprop (including fenoxaprop-P-ethyl), fenoxasulfone, fenpyrazone, fenquinotrione, fentrazamide, flazasulfuron, florasulam, florpyrauxifen (including florpyrauxifen-benzyl), fluazifop (including fluazifop-P-butyl), flucarbazone (including flucarbazone-sodium), flufenacet, flumetsulam, flumioxazin, fluometuron,fomesafen flupyrsulfuron (including flupyrsulfuron-methyl-sodium), fluroxypyr (including fluroxypyr-meptyl), fomesafen, foramsulfuron, glufosinate (including L-glufosinate and the ammonium salts of both), glyphosate (including the diammonium, isopropylammonium and potassium salts thereof), halauxifen (including halauxifen-methyl), haloxyfop (including haloxyfop-methyl), hexazinone, hydantocidin, imazamox (including R-imazamox), imazapic, imazapyr, imazethapyr, indaziflam, iodosulfuron (including iodosulfuron-methyl-sodium), iofensulfuron (including iofensulfuron-sodium), ioxynil, isoproturon, isoxaflutole, lancotrione, MCPA, MCPB, mecoprop-P, mesosulfuron (including mesosulfuron-methyl), mesotrione, metamitron, metazachlor, methiozolin, metolachlor, metosulam, metribuzin, metsulfuron, napropamide, nicosulfuron, norflurazon, oxadiazon, oxasulfuron, oxyfluorfen, paraquat dichloride, pendimethalin, penoxsulam, phenmedipham, picloram, pinoxaden, pretilachlor, primisulfuron-methyl, prometryne, propanil, propaquizafop, propyrisulfuron, propyzamide, prosulfocarb, prosulfuron, pyraclonil, pyraflufen (including pyraflufen-ethyl), pyrasulfotole, pyridate, pyriftalid, pyrimisulfan, pyroxasulfone, pyroxsulam, quinclorac, quinmerac, quizalofop (including quizalofop-P-ethyl and quizalofop-P-tefuryl), rimisoxafen, rimsulfuron, saflufenacil, sethoxydim, simazine, S-metalochlor, sulfentrazone, sulfosulfuron, tebuthiuron, tefuryltrione, tembotrione, terbuthylazine, terbutryn, tetflupyrolimet, thiencarbazone, thifensulfuron, tiafenacil, tolpyralate, topramezone, tralkoxydim, triafamone, triallate, triasulfuron, tribenuron (including tribenuron-methyl), triclopyr, trifloxysulfuron (including trifloxysulfuron-sodium), trifludimoxazin, trifluralin, triflusulfuron, tripyrasulfone, 3-(2-chloro-4-fluoro-5-(3-methyl-2,6-dioxo-4-trifluoromethyl-3,6-dihydropyrimidin-1(2H)—yl)phenyl)-5-methyl-4,5-dihydroisoxazole-5-carboxylic acid ethyl ester,4-hydroxy-1-methoxy-5-methyl-3-[4-(trifluoromethyl)-2-pyridyl]imidazolidin-2-one, 4-hydroxy-1,5-dimethyl-3-[4-(trifluoromethyl)-2-pyridyl]imidazolidin-2-one, 5-ethoxy-4-hydroxy-1-methyl-3-[4-(trifluoromethyl)-2-pyridyl]imidazolidin-2-one, 4-hydroxy-1-methyl-3-[4-(trifluoromethyl)-2-pyridyl]imidazolidin-2-one, 4-hydroxy-1,5-dimethyl-3-[1-methyl-5-(trifluoromethyl)pyrazol-3-yl]imidazolidin-2-one, (4R)1-(5-tert-butylisoxazol-3-yl)-4-ethoxy-5-hydroxy-3-methyl-imidazolidin-2-one, 4-amino-3-chloro-5-fluoro-6-(7-fluoro-1H-indol-6-yl)pyridine-2-carboxylic acid (including agrochemically acceptable esters thereof, for example, methyl 4-amino-3-chloro-5-fluoro-6-(7-fluoro-1H-indol-6-yl)pyridine-2-carboxylate, prop-2-ynyl 4-amino-3-chloro-5-fluoro-6-(7-fluoro-1H-indol-6-yl)pyridine-2-carboxylate and cyanomethyl 4-amino-3-chloro-5-fluoro-6-(7-fluoro-1H-indol-6-yl)pyridine-2-carboxylate), 3-ethylsulfanyl-N-(1,3,4-oxadiazol-2-yl)-5-(trifluoromethyl)-[1,2,4]triazolo[4,3-a]pyridine-8-carboxamide, 3-(isopropylsulfanylmethyl)-N-(5-methyl-1,3,4-oxadiazol-2-yl)-5-(trifluoromethyl)—[1,2,4]triazolo[4,3-a]pyridine-8-carboxamide, 3-(isopropylsulfonylmethyl)-N-(5-methyl-1,3,4-oxadiazol-2-yl)-5-(trifluoromethyl)-[1,2,4]triazolo[4,3-a]pyridine-8-carboxamide, 3-(ethylsulfonylmethyl)-N-(5-methyl-1,3,4-oxadiazol-2-yl)-5-(trifluoromethyl)—[1,2,4]triazolo[4,3-a]pyridine-8-carboxamide, ethyl-2-[[3-[[3-chloro-5-fluoro-6-[3-methyl-2,6-dioxo-4-(trifluoromethyl)pyrimidin-1-yl]-2-pyridyl]oxy]acetate,6-chloro-4-(2,7-dimethyl-1-naphthyl)-5-hydroxy-2-methyl-pyridazin-3-one, tetrahydrofuran-2-ylmethyl (2R)-2-[(4-amino-3,5-dichloro-6-fluoro-2-pyridyl)oxy]propanoate, tetrahydrofuran-2-ylmethyl (2R)-2-[(4-amino-3,5-dichloro-6-fluoro-2-pyridyl)oxy]propanoate, tetrahydrofuran-2-ylmethyl 2-[(4-amino-3,5-dichloro-6-fluoro-2-pyridyl)oxy]propanoate, 2-[(4-amino-3,5-dichloro-6-fluoro-2-pyridyl)oxy]propanoic acid, 2-fluoro-N-(5-methyl-1,3,4-oxadiazol-2-yl)-3-[(R)-propylsulfinyl]-4-(trifluoromethyl)benzamide, 2-fluoro-N-(5-methyl-1,3,4-oxadiazol-2-yl)-3-propylsulfinyl-4-(trifluoromethyl)benzamide, (2-fluorophenyl)methyl 6-amino-5-chloro-2-(4-chloro-2-fluoro-3-methoxy-phenyl)pyrimidine-4-carboxylate and 6-amino-5-chloro-2-(4-chloro-2-fluoro-3-methoxy-phenyl)pyrimidine-4-carboxylic acid. The mixing partners of the compound of Formula (I) may also be in the form of esters or salts, as mentioned e.g. in The Pesticide Manual, Sixteenth Edition, British Crop Protection Council, 2012.

The compound of Formula (I) can also be used in mixtures with other agrochemicals such as fungicides, nematicides or insecticides, examples of which are given in The Pesticide Manual.

The mixing ratio of the compound of Formula (I) to the mixing partner is preferably from 1: 100 to 1000:1.

The mixtures can advantageously be used in the above-mentioned formulations (in which case “active ingredient” relates to the respective mixture of compound of Formula (I) with the mixing partner).

The compounds or mixtures of the present invention can also be used in combination with one or more herbicide safeners. Examples of such safeners include benoxacor, cloquintocet (including cloquintocet-mexyl), cyprosulfamide, dichlormid, fenchlorazole (including fenchlorazole-ethyl), fenclorim, fluxofenim, furilazole, isoxadifen (including isoxadifen-ethyl), mefenpyr (including mefenpyr-diethyl), metcamifen and oxabetrinil.

Particularly preferred are mixtures of a compound of Formula (I) with cyprosulfamide, isoxadifen-ethyl, cloquintocet-mexyl and/or metcamifen.

The safeners of the compound of Formula (I) may also be in the form of esters or salts, as mentioned e.g. in The Pesticide Manual, 16^{th} Edition (BCPC), 2012.

The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048.

Preferably the mixing ratio of compound of Formula (I) to safener is from 100:1 to 1:10, especially from 20:1 to 1:1.

The present invention still further provides a method of controlling weeds at a locus said method comprising application to the locus of a weed controlling amount of a composition comprising a compound of Formula (I). Moreover, the present invention may further provide a method of selectively controlling weeds at a locus comprising crop plants and weeds, wherein the method comprises application to the locus of a weed controlling amount of a composition according to the present invention. ‘Controlling’ means killing, reducing or retarding growth or preventing or reducing germination. It is noted that the compounds of the present invention show a much-improved selectivity compared to know, structurally similar compounds. Generally the plants to be controlled are unwanted plants (weeds). ‘Locus’ means the area in which the plants are growing or will grow. The application may be applied to the locus pre-emergence and/or postemergence of the crop plant. Some crop plants may be inherently tolerant to herbicidal effects of compounds of Formula (I).

Preferred crop plants include maize, wheat, barley and rice.

The rates of application of compounds of Formula I may vary within wide limits and depend on the nature of the soil, the method of application (pre- or post-emergence; seed dressing; application to the seed furrow; no tillage application etc.), the crop plant, the weed(s) to be controlled, the prevailing climatic conditions, and other factors governed by the method of application, the time of application and the target crop. The compounds of Formula I according to the invention are generally applied at a rate of from 10 to 2500 g/ha, especially from 25 to 1000 g/ha, more especially from 25 to 250 g/ha.

The application is generally made by spraying the composition, typically by tractor mounted sprayer for large areas, but other methods such as dusting (for powders), drip or drench can also be used.

Crop plants are to be understood as also including those crop plants which have been rendered tolerant to other herbicides or classes of herbicides (e.g. ALS-, GS-, EPSPS-, PPO-, HPPD-, -PDS and ACCase-inhibitors) by conventional methods of breeding or by genetic engineering. An example of a crop that has been rendered tolerant to imidazolinones, e.g. imazamox, by conventional methods of breeding is Clearfield®summer rape (canola). Examples of crops that have been rendered tolerant to herbicides by genetic engineering methods include e.g. glyphosate- and glufosinate-resistant maize varieties commercially available under the trade names RoundupReady® and LibertyLink®. The compounds of the present invention may also be used in conjunction with plants disclosed in WO2020/236790.

Crop plants are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK®(Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Garde (maize), NuCOTIN33B®(cotton), Bollgard®(cotton), NewLeaf®(potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crop plants are also to be understood to include those which are obtained by conventional methods of breeding or genetic engineering and contain so-called output traits (e.g. improved storage stability, higher nutritional value and improved flavour).

The compositions can be used to control unwanted plants (collectively, ‘weeds’). The weeds to be controlled may be both monocotyledonous species, for example Agrostis, Alopecurus, Avena, Brachiaria, Bromus, Cenchrus, Cyperus, Digitaria, Echinochloa, Eleusine, Lolium, Monochoria, Rottboellia, Sagittaria, Scirpus, Setaria and Sorghum, and dicotyledonous species, for example Abutilon, Amaranthus, Ambrosia, Chenopodium, Chrysanthemum, Conyza, Galium, lpomoea, Nasturtium, Sida, Sinapis, Solanum, Stellaria, Veronica, Viola and Xanthium.

In a further aspect of the present invention there is provided the use of a compound of Formula (I) as defined herein as a herbicide.

Processes for preparation of compounds of Formula (I) Processes for preparation of compounds, e.g. a compound of formula (I) (which optionally can be an agrochemically acceptable salt thereof), are now described, and form further aspects of the present invention.

A compound of formula 1-3 is a compound of Formula I, wherein Q, R², A and R¹₍ₘ₎ are as defined in formula I, and in which R³¹ is C₁-C₄alkyl and may be prepared by a Suzuki reaction, which involves for example, reacting compounds of formula 1-1,

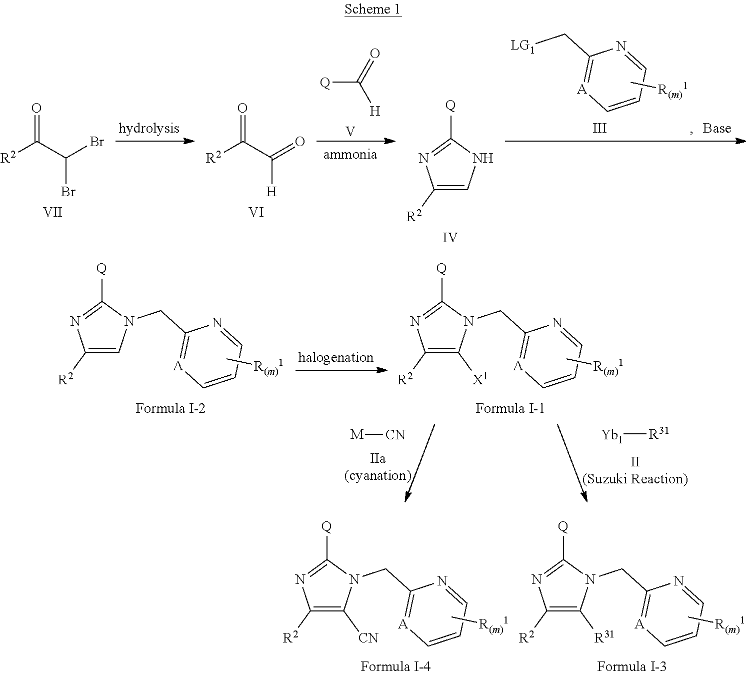

wherein Q, R², A and R¹₍ₘ₎ are as defined in formula I, and wherein X¹ is chlorine, bromine or iodine, with compounds of formula II, wherein R³¹ is C₁-C₄alkyl, and wherein Yb₁ can be a boron-derived functional group, such as for example B(OH)₂ or B(ORb₁)₂ wherein Rb₁ can be a C₁-C₄alkyl group or the two groups ORb₁ can form together with the boron atom a five membered ring, as for example a pinacol boronic ester (scheme 1). The reaction may be catalyzed by a palladium based catalyst, for example tetrakis(triphenyl-phosphine)palladium(0), (1,1′bis(diphenylphosphino) ferrocene)dichloro-palladium-dichloromethane (1:1 complex) or chloro(2-dicyclohexylphosphino-2′,4′,6′-triisopropyl-1,1′-biphenyl)[2-(2′-amino-1,1′-biphenyl)]palladium(II) (XPhos palladacycle), in presence of a base, like sodium carbonate, tripotassium phosphate or cesium fluoride, in a solvent or a solvent mixture, like, for example dioxane, acetonitrile, N,N-dimethyl-formamide, a mixture of 1,2-dimethoxyethane and water or of dioxane/water, or of toluene/water, preferably under inert atmosphere. The reaction temperature can preferentially range from room temperature to the boiling point of the reaction mixture, or the reaction may be performed under microwave irradiation. Such Suzuki reactions are well known to those skilled in the art.

A compound of Formula I-4 is a compound of Formula I where R³ in formula I is CN and may be prepared from compounds of formula 1-1, by reaction with M-CN IIa (cyanation), wherein M is a metal coordinated to the cyanide. Examples of cyanating reagent include NaCN, Zn(CN)₂, or potassium ferrocyanide amongst others. The reaction may be catalyzed by a palladium based catalyst, for example tetrakis(triphenylphosphine)palladium(0), (1,1′bis(diphenylphosphino)ferrocene) dichloro-palladium-dichloromethane (1:1 complex) or chloro(2-dicyclohexylphosphin o-2′,4′,6′-triisopropyl-1,1′-biphenyl)[2-(2′-amino-1,1′-biphenyl)]palladium(lI) (XPhos palladacycle), in presence of a base, like sodium carbonate, tripotassium phosphate or cesium fluoride, in a solvent or a solvent mixture, like, for example dioxane, acetonitrile, N,N-dimethyl-formamide, a mixture of 1,2-dimethoxyethane and water or of dioxane/water, or of toluene/water, preferably under inert atmosphere. The reaction temperature can preferentially range from room temperature to the boiling point of the reaction mixture, or the reaction may be performed under microwave irradiation. Such reactions are well known to those skilled in the art.

Compounds of formula 1-1, can be prepared by a halogenation reaction, which involves for example, reacting compounds of formula 1-2, wherein Q, R₂, A and R¹₍ₘ₎ are as defined in formula I, with halogenating reagents such as N-chlorosuccinimide (NCS), N-bromosuccinimide (NBS), or N-iodosuccinimide (NIS), optionally in the presence of an additive, such as for example p-toluenesulfonic acid. Alternatively, the halogenation may involve chlorine, bromine or iodine. Such halogenation reactions are carried out in a suitable solvent, such as chloroform, carbon tetrachloride, 1,2-dichloroethane, acetic acid, diethyl ether, acetonitrile or N,N-dimethylformamide, at temperatures between 20-200° C., preferably room temperature to 100° C.

Compounds of formula 1-2, can be prepared by reacting compounds of formula IV, with reagents of the formula III, wherein A and R¹(m) are as defined in formula I, and in which LG₁ is a halogen, preferably iodine, bromine or chlorine (or a pseudo-halogen leaving group, such as a (halo)alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, optionally in the presence of potassium iodide in an inert solvent such as tetrahydrofuran, dioxane, water, N,N-dimethylformamide DMF, N,N-dimethylacetamide or acetonitrile and the like, at temperatures between 0 and 120° C., by procedures well known to those skilled in the art.

Alternatively, compounds of formula 1-2 can be prepared following scheme 1a. In scheme 1a, compounds of formula 1-2 are prepared from compounds of formula IVaa,

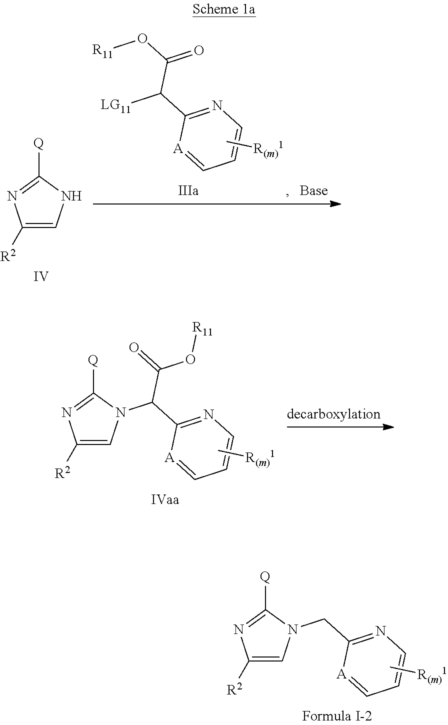

wherein R¹¹ is Cr—C₄alkyl, via decarboxylation reaction. Such reactions can be carried out in the presence of a base such as sodium hydroxide, potassium hydroxide, potassium carbonate and similar others. Compounds of formula IVaa can be prepared by reacting compounds of formula IV with compounds of formula IIIa, wherein LG11 is a leaving group like chloro or bromo in the presence of a base such as potassium carbonate and in the presence of a solvent such as acetonitrile, tetrahydrofuran, sulfolane and at temperature between room temperature to the boiling point of the solvent and optionally under microwave irradiation.

Compounds of formula IV can be prepared by condensation reaction of compounds of formula VI or its hydrated form, with compounds of formula V, in the presence of ammonia or its surrogates such as ammonium hydroxide. The reaction can be carried out in the presence of solvent such as methanol, tetrahydrofuran, ethanol, amongst others and at temperatures between 20-200° C., preferably room temperature to 100° C. Compounds of formula VI or its hydrated form, wherein R² is as defined in formula I can be prepared by hydrolysis of compounds of formula VII, wherein R² is as defined in formula I. Such two step reactions are well known in literature.

A compound of formula 1-5 is a compound of Formula I, wherein Q, A and R¹₍ₘ₎ are as defined in formula I, and in which R³ in formula I is H and R² in formula I is a halogen defined as X² (Scheme 2).

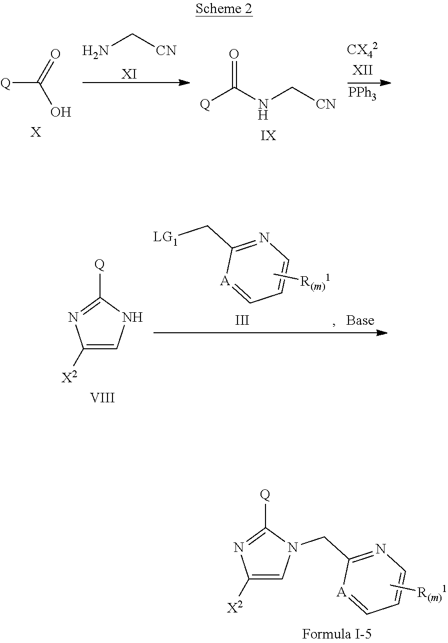

Compounds of formula 1-5 can be prepared by reacting compounds of formula VIII, with reagents of the formula III, wherein A and R¹₍ₘ₎ are as defined in formula I, and in which LG₁ is a halogen, preferably iodine, bromine or chlorine (or a pseudo-halogen leaving group, such as a (halo)alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, optionally in the presence of potassium iodide in an inert solvent such as tetrahydrofuran, dioxane, water, N,N-dimethylformamide DMF, N,N-dimethylacetamide or acetonitrile and the like, at temperatures between 0 and 120° C., by procedures well known to those skilled in the art.

Compounds of formula VIII can be prepared by reacting compounds of formula IX with CX²₄ XII, wherein X² is a halogen, preferably chlorine or bromine in the presence of triphenyl phosphine. The reaction can be carried out in the presence of solvent such as acetonitrile, carbon tetrachloride or tetrahydrofuran and at temperature between room temperature and boiling point of solvent. Such reactions are known in the literature and for example described in Org. Lett. 2004, 6, 6, 929-931.

Compounds of formula IX can be prepared by amide coupling of compounds of formula X and compounds of formula XI. Reaction can be carried out using amide coupling reagent such as 1-propanephosphonic anhydride, (1-[Bis(dimethylamino)methylene]-1H-1,2,3-triazolo[4,5-b]pyridinium 3-oxide hexafluoro phosphate, amongst others optionally in the presence of base such as pyridine, triethyl amine amongst others. Such reactions are well known to those skilled in the art and described in literature.

A compound of formula 1-6 is a compound of Formula I, wherein Q, A and R¹₍ₘ₎ are as defined in formula I, and in which R² in formula I is halogen and defined as X⁴ (scheme 3).

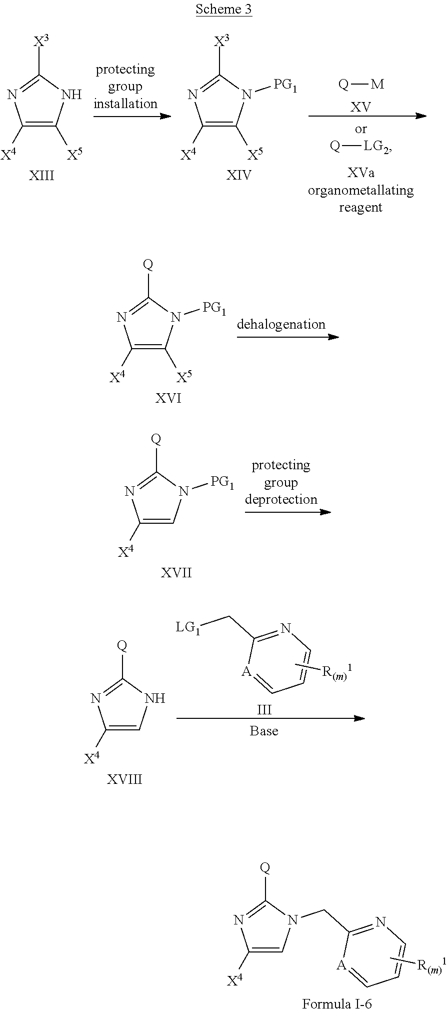

Compounds of formula 1-6 can be prepared by reacting compounds of formula XVIII, with reagents of the formula III, wherein A and R¹₍ₘ₎ are as defined in formula I, and in which LG₁ is a halogen, preferably iodine, bromine or chlorine (or a pseudo-halogen leaving group, such as a (halo)alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, optionally in the presence of potassium iodide in an inert solvent such as tetrahydrofuran, dioxane, water, N,N-dimethylformamide DMF, N,N-dimethylacetamide or acetonitrile and the like, at temperatures between 0 and 120° C., by procedures well known to those skilled in the art.

Compounds of formula XVIII, wherein Q is as defined in formula I, and X⁴ is halogen, can be prepared by protecting group deprotection reaction from compounds of formula XVII, wherein PG₁ is a N-protecting group for example acetyl, trimethylsilylethoxymethyl (SEM), tert-butyloxycarbonyl amongst others amino protecting groups. Such reactions are well known to those skilled in the art and can be carried out for example using base catalyzed or acid catalyzed such as HCl. Compounds of formula XVII can be prepared by selective dehalogenation reaction from compounds of formula XVI, wherein X⁴ and X⁵ are halogens. Such reactions can be carried out by metal halogen exchange using organometallic reagents such as butyl lithium, Grignard reagents or organozinc reagents and further quenching the reaction with water or aqueous acidic solution.

Compounds of formula XVI can be prepared by substitution reactions or cross-coupling reaction of compounds of formula XIV with compounds of formula XV, wherein M is alkali metals such as lithium or potassium, alkaline earth metal such as magnesium or transition metal such as copper or zinc. Reaction can optionally be carried out in the presence of metal catalyst such as Palladium or copper and in the presence of ligand such as PPh₃ amongst other phosphine-based ligands, or nitrogen containing ligands for copper such as 1,2-dimethylethylenediamine amongst others. Reaction can be carried out in the presence of solvent such as tetrahydrofuran, dioxane or toluene amongst others. Alternatively compounds of formula XVI can be prepared by substitution reaction which involves metal-halogen exchange of compounds of formula XIV with organometallic reagents such as butyl lithium or Grignard reagents and further reacting then with compounds of formula XVa, wherein LG₂ is a is a halogen, preferably iodine, bromine or chlorine (or a pseudo-halogen leaving group, such as a (halo)alkyl or phenyl sulfonate ester, e.g. triflate), in an inert solvent such as tetrahydrofuran, dioxane, N,N-dimethylformamide DMF, N,N-dimethylacetamide and the like, at temperatures between −80° C. and 120° C., by procedures well known to those skilled in the art.

Compounds of formula XIV can be prepared from compounds of formula XIII by protection group installation. Such reactions can be carried out in the presence of base such as sodium hydride, potassium carbonate, sodium carbonate, and in the presence of suitable protecting group reagents such as 2-(chloromethoxy)ethyl-trimethyl-silane, acetyl chloride, di-tert-butyl dicarbonate and in the presence of solvent such as tetrahydrofuran, methanol, water, acetonitrile, dimethylformamide. Such reactions are well known in the literature.

A compound of formula 1-7 is a compound of Formula I, wherein Q, A and R¹₍ₘ₎ are as defined in formula I, and in which X⁶ and X⁷ are halogens (scheme 4).

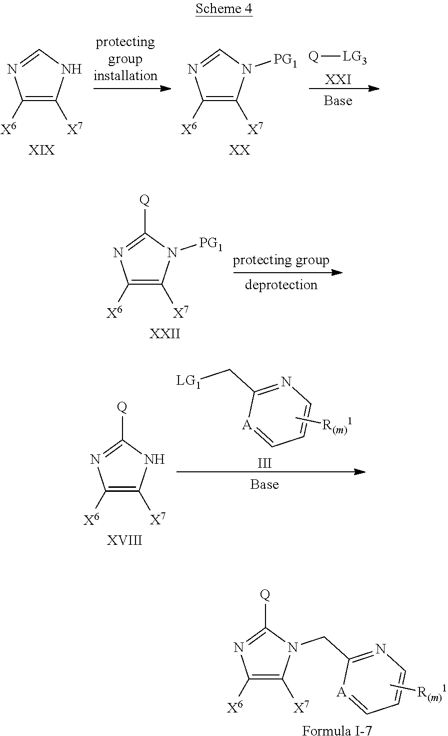

Compounds of formula 1-7 can be prepared from compounds of formula XXIII, wherein X⁶ and X⁷ are halogens following procedure analogous to procedure as described in scheme 3 for the conversion of compounds of formula XVIII to compounds of formula 1-6. Compounds of formula XXIII can be prepared from compounds of formula XXII following procedure analogous to as described in scheme 3 for the conversion of compounds of formula XVII to compounds of formula XVIII. Compounds of formula XXII can be prepared by deprotonation of compounds of formula XX using suitable base such as butyl lithium or lithium diisopropylamide amongst other strong bases, followed by substitution reaction with compounds of formula XXI, wherein LG₃ is a halogen, preferably iodine, bromine or chlorine (or a pseudo-halogen leaving group, such as a (halo)alkyl or phenyl sulfonate ester, e.g. triflate). The reaction is generally carried out in the presence of solvent such as tetrahydrofuran or toluene and at temperature in the range of −80° C. to room temperature. Compounds of formula XX can be prepared from compounds of formula XIX following procedure analogous to as described in scheme 3 for the conversion of compounds of formula XIII to compounds of formula XIV.

Alternatively compounds of formula 1-1 can be prepared following scheme 5. In scheme 5 compounds of formula 1-1 can be prepared from compounds of formula XXVII via fluorination reaction using fluorinating reagents such as diethylaminosulfur trifluoride or bis(2-methoxyethyl)aminosulfur trifluoride amongst others.

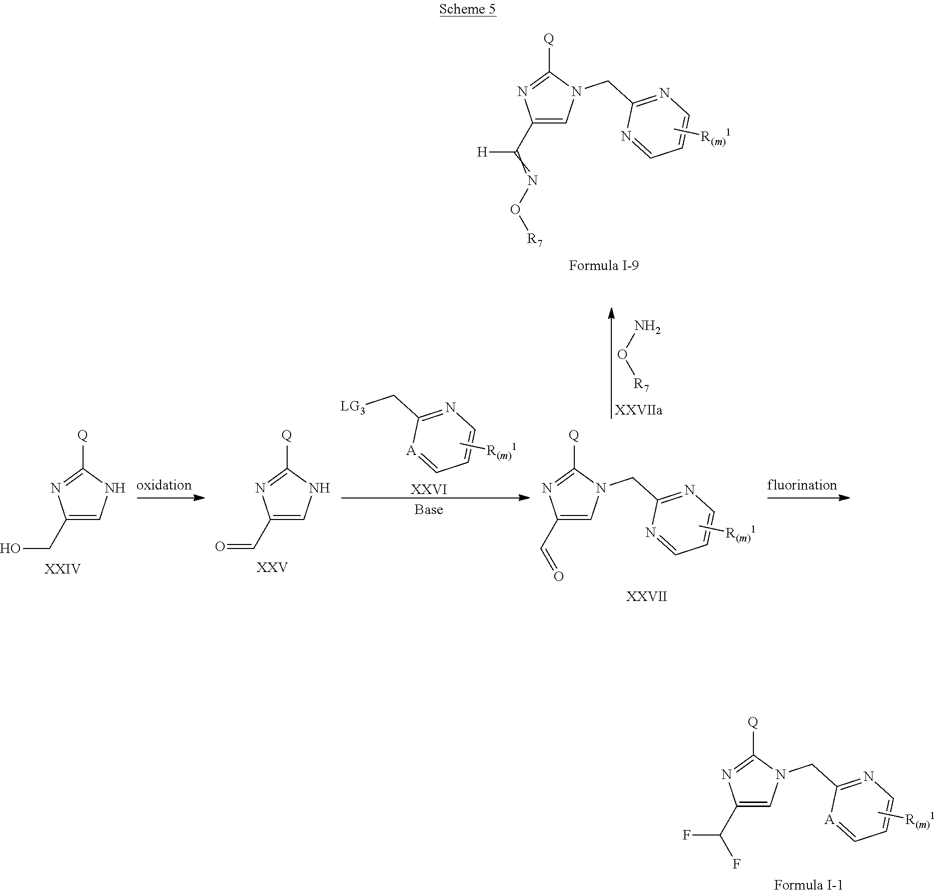

Compounds of formula I, wherein R³ is H and R² is —C(H)═NOR⁷ and wherein R⁷ is as defined in formula I above can be represented by compounds of formula 1-9. Compounds of formula 1-9 can be prepared by the condensation reaction of compounds of formula XXVII with compounds of formula XXVIla. Such reactions are well known in the literature.

Compounds of formula XXVII can be prepared by reacting compounds of formula XXV with compounds of formula XXVI, wherein LG₃ is a halogen, preferably iodine, bromine or chlorine (or a pseudo-halogen leaving group, such as a (halo)alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, optionally in the presence of potassium iodide in an inert solvent such as tetrahydrofuran, dioxane, water, N,N-dimethylformamide DMF, sulfolane, N,N-dimethylacetamide or acetonitrile and the like, at temperatures between 0 and 120° C., by procedures well known to those skilled in the art. Compounds of formula XXV can be prepared from compounds of formula XXIV via oxidation reaction using oxidizing agents such as MnO₂, SO₃.pyridine, pyridinium dichromate or pyridinium chlorochromate amongst other alcohol oxidizing reagents. Compounds of formula XXIV can be prepared following procedure reported in literature for example in J. Med. Chem. 1995, 38, 2251-2255.

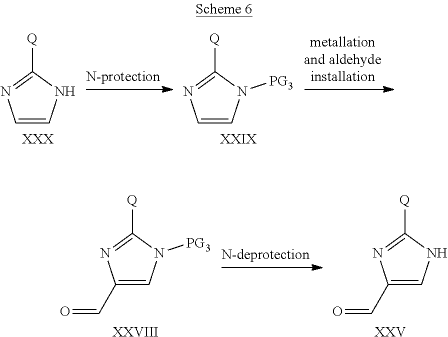

Alternatively compounds of formula XXV, can be prepared from compounds of formula XXVIII, wherein PG₃ is a N-protection group like N,N-dimethylsulfamoyl, acetyl, tert-Butyloxycarbonyl and similar others via N-deprotection reaction. Such deprotection reactions can be carried out in the presence of an acid catalyst such as hydrochloric acid or base such as sodium hydroxide or potassium carbonate. Compounds of formula XXVIII, can be prepared from compounds of formula XXIX via metallation reaction using strong base such as n-butyl lithium, lithium diisopropylamide and subsequently reacting with N,N-dimethylformamide to install the aldehyde. Compounds of formula XXIX can be prepared from compounds of formula XXX via N-protection reaction. Examples of reagents that can be utilized for such N-protection reactions are di-tert-butyl decarbonate, acetyl chloride, acetic anhydride, N,N-dimethylsulfamoyl chloride and similar others.

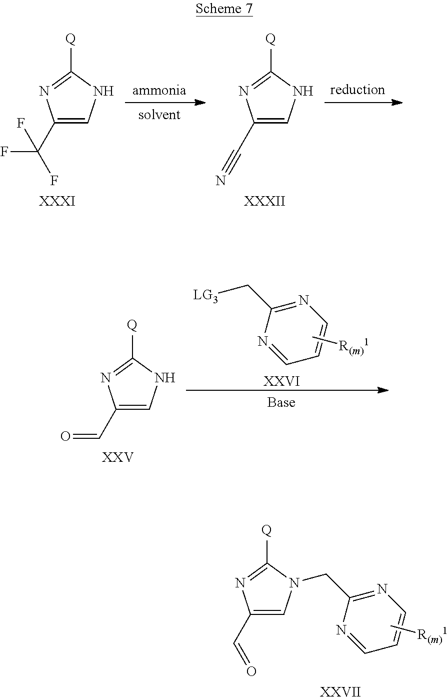

Alternatively compounds of formula XXVII can be prepared following scheme 7. In scheme 7, compound of formula XXVII can be prepared by reacting compounds of formula XXV with compounds of formula XXVI, following procedure as described in scheme 5. Compound of formula XXV can be prepared by reacting compound of formula XXXII with a suitable reducing agent such as diisobutyl aluminium hydride. Compound of formula XXXII can be prepared by reacting compound of formula XXXI with ammonium hydroxide or similar other ammonia surrogates to transform the trifluoromethyl group to a cyano group. Such reactions are well documented in the literature (see for example Matthews, D. P.; Whitten, J. P.; McCarthy, J. R. J. Org. Chem. 1986, 51, 3228). Synthesis of imidazole compounds of Formula XXXI are well documented in the literature (see for example Journal of Medicinal Chemistry, 2000, 43, 2165 and Synthetic Communications, 2020, 50, 700).

The following non-limiting examples provide specific synthesis methods for representative compounds of the present invention, as referred to in the Table below.

### Example 1: Preparation of 5-chloro-2-[[4-chloro-2-(4,4,4-trifluorobutyl)imidazol-1-yl]methyl]pyrimidine (Compound 1.001)

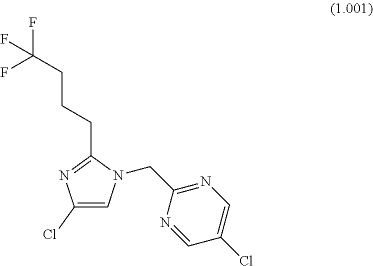 [SMILES: FC(F)(F)CCCc1nc(Cl)cn1Cc1ncc(Cl)cn1]

Step 1: Preparation of N-(cyanomethyl)-5,5,5-trifluoro-pentanamide (11)

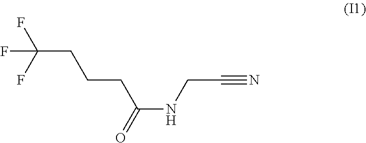 [SMILES: N#CCNC(=O)CCCC(F)(F)F]

To a suspension of 5,5,5-trifluoropentanoic acid (620 mg, 3.97 mmol), aminoacetonitrile hydrochloride (368 mg, 3.97 mmol) and triethylamine (1.11 mL, 7.94 mmol) in ethyl acetate (I0 mL), was added 1-propanephosphonic anhydride (50 mass % in ethyl acetate; 3.07 mL, 5.16 mmol) and this mixture was stirred at room temperature overnight. The reaction mixture was diluted with water (50 mL) and extracted with ethyl acetate (3×50 mL). The organics were combined, washed with saturated aqueous sodium bicarbonate solution (2×100 mL), then brine (100 mL), dried over magnesium sulphate and concentrated to give crude product. The crude material was subjected to column chromatography on silica gel using 0-100% ethyl acetate in cyclohexane to give N-(cyanomethyl)-5,5,5-trifluoro-pentanamide 11 (210 mg, 27%) as an orange crushed foam. ¹H NMR (400 MHz, CDCl₃) δ=5.90 (br s, 1H), 4.20 (d, 2H), 2.32 (t, 2H), 2.24-2.10 (m, 2H), 2.00-1.89 (m, 2H)

Step 2: Preparation of 4-chloro-2-(4,4,4-trifluorobutyl)-1H-imidazole (I2)

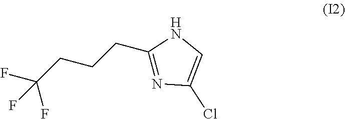 [SMILES: FC(F)(F)CCCc1nc(Cl)c[nH]1]

Carbon tetrachloride (0.39 mL, 4.00 mmol) was added to a stirred suspension of N-(cyanomethyl)-5,5,5-trifluoro-pentanamide 11 (210 mg, 1.08 mmol) and triphenyl phosphine (1.07 g, 4.00 mmol) in acetonitrile (5 mL) under nitrogen. The reaction mixture was heated at 50° C. for 1 hour then allowed to cool to room temperature. The reaction mixture was diluted with aqueous 2M sodium hydroxide (50 mL) and then extracted with ethyl acetate (3×50 mL). The organics were combined, washed with brine (100 mL), dried over magnesium sulphate and concentrated to give crude product. The crude material was subjected to column chromatography on silica gel using 0-100% ethyl acetate in cyclohexane to give 4-chloro-2-(4,4,4-trifluorobutyl)-1H-imidazole 12 (88 mg, purity 70%). ¹H NMR (400 MHz; CDCl₃) δ=6.69 (s, 1H), 2.63 (t, 2H), 2.08-1.92 (m, 2H), 1.92-1.80 (m, 2H)

Step 3: Preparation of 5-chloro-2-[[4-chloro-2-(4,4,4-trifluorobutyl)imidazol-1-yl]methyl]pyrimidine (1.001)

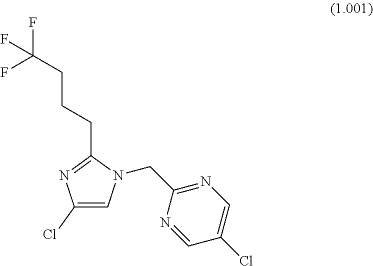 [SMILES: FC(F)(F)CCCc1nc(Cl)cn1Cc1ncc(Cl)cn1]

4-Chloro-2-(4,4,4-trifluorobutyl)-1H-imidazole 12 (88 mg), 5-chloro-2-(chloromethyl)pyrimidine hydrochloride (103 mg, 0.52 mmol), potassium carbonate (144 mg, 1.03 mmol), potassium iodide (6.9 mg, 0.041 mmol), acetonitrile (4 ml) and water (0.13 ml) were heated at 80° C. overnight. The reaction mixture was allowed to cool to room temperature then was diluted with water (50 mL) and extracted with ethyl acetate (3×50 mL). The organics were combined, washed with brine (100 mL), dried over magnesium sulphate and concentrated to give crude product. The crude material was subjected to column chromatography on silica gel using 0-50% ethyl acetate in cyclohexane to give 5-chloro-2-[[4-chloro-2-(4,4,4-trifluorobutyl)imidazol-1-yl]methyl]pyrimidine 1.001 (50 mg, 14% over 2 steps). 1 H NMR (400 MHz; CDCl₃) δ=8.59 (s, 2H), 6.77 (s, 1H), 5.10 (s, 2H), 2.69 (t, 2H), 2.19-2.03 (m, 2H), 2.00-1.89 (m, 2H)

### Example 2: Preparation of 5-chloro-2-[[4-(trifluoromethyl)-2-(3,3,3-trifluoropropyl)imidazol-1-yl]methyl]pyrimidine (Compound 1.002)

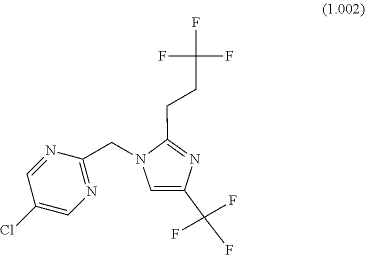 [SMILES: FC(F)(F)CCc1nc(C(F)(F)F)cn1Cc1ncc(Cl)cn1]

Step 1: Preparation of 3,3,3-trifluoro-2-oxo-propanal (13)

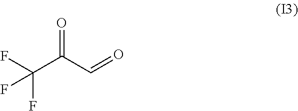

To a 50 mL flask was added sodium acetate (508 mg, 6.13 mmol) and water (2.4 mL). 1,1-Dibromo-3,3,3-trifluoroacetone (410 pL, 2.86 mmol) was added to the above solution giving a turbid solution/suspension. The reaction mixture was then heated to 100° C. for 30 minutes. The reaction mixture was then cooled to room temperature to provide a solution of 13 which was used as such for the next step.

Step 2: Preparation of 4-(trifluoromethyl)-2-(3,3,3-trifluoropropyl)-1H-imidazole (I4)

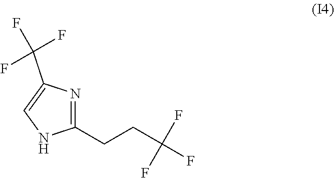 [SMILES: FC(F)(F)CCc1nc(C(F)(F)F)c[nH]1]

To the solution of 13 prepared in step 1 was added 35% aqueous ammonia (2.4 mL, 21 mmol) followed by a solution of 4,4,4-trifluorobutyraldehyde (275 mg, 2.18 mmol) in methanol (2.4 mL). The mixture was stirred at room temperature for 17 hours. The mixture was diluted with water and extracted with ethyl acetate. The combined organics were concentrated and subjected to column chromatography on silica gel using 0-70% ethyl acetate in cyclohexane to give 4-(trifluoromethyl)-2-(3,3,3-trifluoropropyl)-1H-imidazole (I4) as a white solid (473 mg, 89% yield). ¹H NMR (400 MHz, CDC3) δ=9.18 (br s, 1H), 7.31 (s, 1H), 3.05-2.98 (m, 2H), 2.74-2.59 (m, 2H)

Step 3: Preparation of 5-chloro-2-[[4-(trifluoromethyl)-2-(3,3,3-trifluoropropyl)imidazol-1-yl]methyl]pyrimidine (1.002)

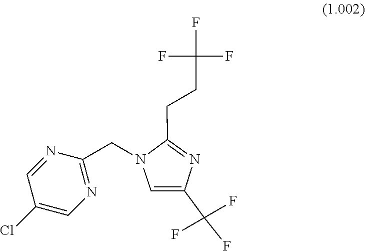 [SMILES: FC(F)(F)CCc1nc(C(F)(F)F)cn1Cc1ncc(Cl)cn1]

To a 50 mL flask was added 4-(trifluoromethyl)-2-(3,3,3-trifluoropropyl)-1H-imidazole 14 (155 mg, 0.634 mmol), potassium carbonate (241 mg, 1.745 mmol), 5-chloro-2-(chloromethyl)pyrimidine hydrochloride (162 mg, 0.772 mmol) and potassium iodide (33 mg, 0.199 mmol). The mixture was treated with acetonitrile (2.0 mL) and water (100 pL), heated to 70° C. and stirred for 1 hour. On completion, the mixture was allowed to cool before being diluted with water and extracted with ethyl acetate. The combined organics were concentrated and subjected to reverse-phase column chromatography on C-18 silica gel using 50-100% acetonitrile in water, both with 0.1% formic acid, to give 5-chloro-2-[[4-(trifluoromethyl)-2-(3,3,3-trifluoropropyl)imidazol-1-yl]methyl]pyrimidine 1.002 as a beige solid (177 mg, 74% yield). ¹H NMR (400 MHz, CDC3) δ=8.69 (s, 2H), 7.34-7.31 (m, 1H), 5.27 (s, 2H), 3.04-2.94 (m, 2H), 2.82-2.64 (m, 2H)

### Example 3: Preparation of 2-[[4-bromo-2-(4,4,4-trifluorobutyl)imidazol-1-yl]methyl]-5-chloro-pyrimidine (Compound 1.003)

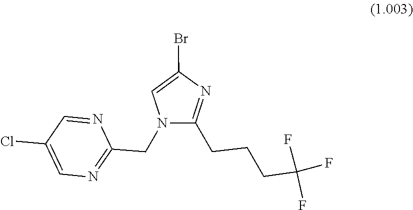 [SMILES: FC(F)(F)CCCc1nc(Br)cn1Cc1ncc(Cl)cn1]

Step 1 —Preparation of trimethyl-[2-[(2,4,5-tribromoimidazol-1-yl)methoxy]ethyl]silane (I5):

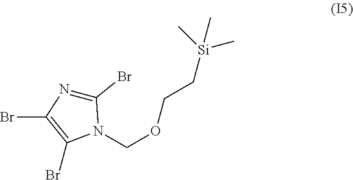 [SMILES: C[Si](C)(C)CCOCn1c(Br)nc(Br)c1Br]

To a rapidly stirred suspension of 2,4,5-tribromoimidazole (5.00 g, 16.4 mmol) and potassium carbonate (6.80 g, 49.2 mmol) in acetonitrile (45 mL) was added 2-(chloromethoxy)ethyl-trimethyl-silane (4.3 mL, 25 mmol) in a single portion. The reaction was stirred at room temperature for 16 h and then quenched with saturated NH₄Cl (50 mL). Ethyl acetate (50 mL) was added, and the phases were separated.

The aqueous phase was extracted with ethyl acetate (3×30 mL), the organics combined, washed with saturated brine solution (50 mL) and then concentrated onto granulated celite. The crude product was purified by flash chromatography on silica gel using a gradient of 0-30% ethyl acetate in cyclohexane as eluent to give trimethyl-[2-[(2,4,5-tribromoimidazol-1-yl)methoxy]ethyl]silane I5 (6.78 g, 95%) as a white solid. ¹H NMR (400 MHz, CDCl₃) δ=5.34 (s, 2H), 3.69-3.54 (m, 2H), 1.01-0.89 (m, 2H), 0.02 (s, 9H)

Step 2 —Preparation of 2-[[4,5-dibromo-2-(4,4,4-trifluorobutyl)imidazol-1-yl]methoxy]ethyl-trimethyl-silane (I6):

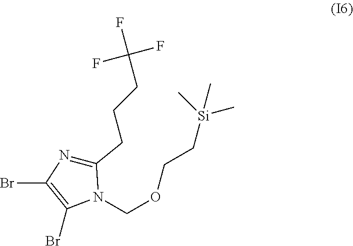 [SMILES: C[Si](C)(C)CCOCn1c(CCCC(F)(F)F)nc(Br)c1Br]

A solution of trimethyl-[2-[(2,4,5-tribromoimidazol-1-yl)methoxy]ethyl]silane I5 (0.50 g, 1.15 mmol) in THF (10 mL) was cooled to −70° C. under N₂ and n-BuLi (2.5 M in hexanes) (0.60 mL, 1.5 mmol) was added dropwise maintaining a constant temperature below −60° C. The reaction mixture was then stirred at −70° C. for 0.5 h. To the reaction mixture was added 1,1,1-trifluoro-4-iodobutane (0.22 mL, 1.73 mmol) dropwise. The reaction was stirred at −70° C. under N₂ for 1 h and then allowed to warm slowly to room temperature. Stirring was continued at room temperature under N₂ for 4 h. The reaction was quenched with saturated brine solution (20 mL) and ethyl acetate (20 mL) was added. The phases were separated and the aqueous extracted with ethyl acetate (3×10 mL). The organics were combined and concentrated onto granulated celite. The crude product was purified by flash chromatography on silica gel using a gradient of 0-35% ethyl acetate in cyclohexane as eluent to give 2-[[4,5-dibromo-2-(4,4,4-trifluorobutyl)imidazol-1-yl]methoxy]ethyl-trimethyl-silane I6 (0.25 g, 47%) as a pale yellow oil. ¹HNMR (400 MHz, chloroform) δ=5.25 (s, 2H), 3.58-3.53 (m, 2H), 2.86-2.77 (m, 2H), 2.27-2.15 (m, 2H), 2.12-2.00 (m, 2H), 0.93-0.88 (m, 2H), 0.00 (m, 9H)

### Step 3 —Preparation of 2-[[4-bromo-2-(4,4,4-trifluorobutyl)imidazol-1-yl]methoxy]ethyl-trimethyl-silane (I7)

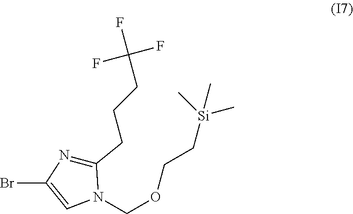 [SMILES: C[Si](C)(C)CCOCn1cc(Br)nc1CCCC(F)(F)F]

To a solution of 2-[[4,5-dibromo-2-(4,4,4-trifluorobutyl)imidazol-1-yl]methoxy]ethyl-trimethyl-silane I6 (0.25 g, 0.54 mmol) in THF (7 mL) was cooled to −70° C. under N₂ and n-BuLi (2.5 M in hexanes, 0.30 mL, 0.75 mmol) was added dropwise maintaining a constant temperature below −60° C. The reaction mixture was then stirred at −70° C. for 0.5 h. To the reaction mixture was added water (1.00 mL). The reaction was stirred at −70° C. under N₂ for 1 h and then allowed to warm slowly to room temperature. The reaction mixture was concentrated onto granulated celite. The crude product was purified by flash chromatography on silica gel using a gradient of 0-30% ethyl acetate in cyclohexane as eluent to give 2-[[4-bromo-2-(4,4,4-trifluorobutyl)imidazol-1-yl]methoxy]ethyl-trimethyl-silane I7 (72 mg, 35%) as a colourless oil.

¹H NMR (400 MHz, CDCl₃) δ=6.96-6.84 (m, 1H), 5.16 (s, 2H), 3.66-3.37 (m, 2H), 2.79 (t, 2H), 2.34-2.14 (m, 2H), 2.13-2.01 (m, 2H), 1.01-0.80 (m, 2H), 0.01 (s, 9H)

Step 4 —Preparation of 4-bromo-2-(4,4,4-trifluorobutyl)-1H-imidazole;hydrochloride (I8):

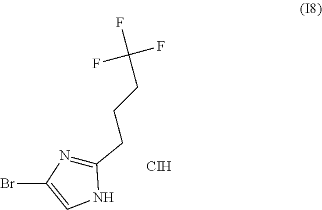

To a solution of 2-[[4-bromo-2-(4,4,4-trifluorobutyl)imidazol-1-yl]methoxy]ethyl-trimethyl-silane I7 (96 mg, 0.25 mmol) in ethanol (2 mL) was added 6M HCl (0.6 mL, 3.6 mmol) and the reaction mixture was heated to 70° C. for 6 h. The reaction mixture was concentrated under vacuum to give 4-bromo-2-(4,4,4-trifluorobutyl)-1H-imidazole;hydrochloride I8 (71 mg, 97%) as a pale yellow solid. ¹H NMR (400 MHz, MeOD) δ=7.60-7.52 (m, 1H), 7.69-7.42 (m, 1H), 3.10-2.95 (m, 2H), 2.41-2.20 (m, 2H), 2.13-1.95 (m, 2H)

Step 5 —Preparation of 2-[[4-bromo-2-(4,4,4-trifluorobutyl)imidazol-1-yl]methyl]-5-chloro-pyrimidine (Compound 1.003):

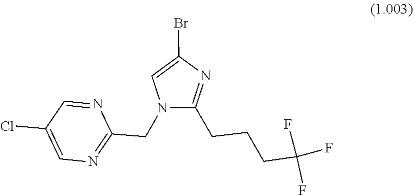 [SMILES: FC(F)(F)CCCc1nc(Br)cn1Cc1ncc(Cl)cn1]

To a stirred suspension of 4-bromo-2-(4,4,4-trifluorobutyl)-1H-imidazole;hydrochloride I8 (0.071 g, 0.24 mmol), potassium iodide (9 mg, 0.05 mmol) and potassium carbonate (0.117 g, 0.85 mmol) in acetonitrile (5 mL) and H₂O (0.5 mL) was added 5-chloro-2-(chloromethyl)pyrimidine hydrochloride (0.068 g, 0.32 mmol) in a single portion. The reaction was then heated to 80° C. under N₂ for 16h.

The reaction was cooled to room temperature and concentrated onto granulated celite. The crude product was purified by flash chromatography on silica gel using a gradient of 0-70% ethyl acetate in cyclohexane as eluent to give 2-[[4-bromo-2-(4,4,4-trifluorobutyl)imidazol-1-yl]methyl]-5-chloro-pyrimidine 1.003 (76 mg, 82%) as a pale yellow oil. ¹H NMR (400 MHz, CDCl₃) δ=8.67 (s, 2H), 6.93 (s, 1H), 5.20 (s, 2H), 2.78 (t, 2H), 2.29-2.10 (m, 2H), 2.09-1.97 (m, 2H)

### Example 4: Preparation of 5-chloro-2-[[4,5-dichloro-2-(4,4,4-trifluorobutyl)imidazol-1-yl]methyl]pyrimidine (Compound 1.004)

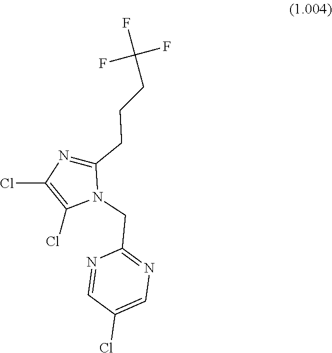 [SMILES: FC(F)(F)CCCc1nc(Cl)c(Cl)n1Cc1ncc(Cl)cn1]

Step 1 —Preparation of 2-[(4,5-dichloroimidazol-1-yl)methoxy]ethyl-trimethyl-silane I(9):

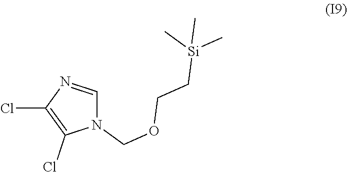

To a solution of 4,5-dichloro-1H-imidazole (1.05 g, 7.67 mmol), in acetonitrile (10 mL) was added potassium carbonate (2.52 g, 18.2 mmol) at room temperature. The solution turned yellow and 2-(chloromethoxy)ethyl-trimethyl-silane (1.70 mL, 9.7 mmol) was added in a single portion turning the reaction mixture beige. The reaction was stirred at room temperature for 1 h then concentrated onto granulated celite. The crude product was purified by flash chromatography on silica gel using a gradient of 0-100% ethyl acetate in cyclohexane as eluent to give 2-[(4,5-dichloroimidazol-1-yl)methoxy]ethyl-trimethyl-silane I9 (1.76 g, 86%) as a pale yellow solid. ⁴H NMR (400 MHz, CDCl₃) δ=7.51 (s, 1H), 5.26 (s, 2H), 3.56 (dd, 2H), 1.04-0.76 (m, 2H), 0.01 (s, 9H)

Step 2 —Preparation of 2-[[4,5-dichloro-2-(4,4,4-trifluorobutyl)imidazol-1-yI]methoxy]ethyl-trimethyl-silane (I10):

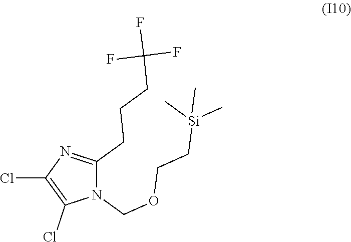 [SMILES: C[Si](C)(C)CCOCn1c(CCCC(F)(F)F)nc(Cl)c1Cl]

A solution of 2-[(4,5-dichloroimidazol-1-yl)methoxy]ethyl-trimethyl-silane I9 (0.50 g, 1.87 mmol) in tetrahydrofuran (10 mL) was cooled to −70° C. under N₂ and n-BuLi (2.5 M in hexanes) (1.00 mL, 2.5 mmol) was added dropwise maintaining a constant temperature below −60° C. The reaction mixture was then stirred at −70° C. for 0.5 h. To the reaction mixture was added 1,1,1-trifluoro-4-iodobutane (0.36 mL, 2.8 mmol) dropwise. The reaction was stirred at −70° C. under N₂ for 1 h and allowed to warm slowly to room temperature. Stirring was continued at room temperature under N₂ for 16 h. The reaction was quenched with methanol (10 mL) and the reaction mixture concentrated onto granulated celite. The crude product was purified by flash chromatography on silica gel using a gradient of 0-20% ethyl acetate in cyclohexane as eluent to give 2-[[4,5-dichloro-2-(4,4,4-trifluorobutyl)imidazol-1-yl]methoxy]ethyl-trimethyl-silane I10 (0.43 g, 60%) as a pale yellow oil. ¹H NMR (400 MHz, CDCl₃) 6=5.21 (s, 2H), 3.60-3.50 (m, 2H), 2.79 (t, 2H), 2.31-2.14 (m, 2H), 2.14-1.99 (m, 2H), 0.95-0.86 (m, 2H), 0.00 (s, 9H)

Step 3 —Preparation of 4,5-dichloro-2-(4,4,4-trifluorobutyl)-1H-imidazole (I11):

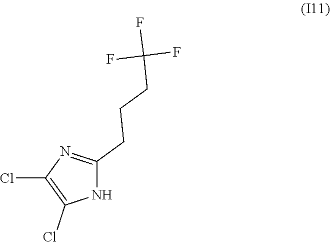 [SMILES: FC(F)(F)CCCc1nc(Cl)c(Cl)[nH]1]

To a solution of 2-[[4,5-dichloro-2-(4,4,4-trifluorobutyl)imidazol-1-yl]methoxy]ethyl-trimethyl-silane I10 (0.31 g, 0.82 mmol) in ethanol (5 mL) was added 6M HCl (2.5 mL, 15 mmol) and the reaction was heated to 60° C. for 2 h. The reaction mixture was cooled to room temperature then concentrated onto granulated celite. The crude product was purified by flash chromatography on C₁₈ reverse-phase silica using a gradient of 50-100% acetonitrile in water (with 0.1% formic acid modifier) as eluent to give 4,5-dichloro-2-(4,4,4-trifluorobutyl)-1H-imidazole 111 (65 mg, 32%) as a white solid. ¹H NMR (400 MHz, MeOD) 5=2.70 (t, 2H), 2.34-2.10 (m, 2H), 1.92 (quin, 2H)

Step 4 —Preparation of 5-chloro-2-[[4,5-dichloro-2-(4,4,4-trifluorobutyl)imidazol-1-yIl]methyl]pyrimidine 1.004:

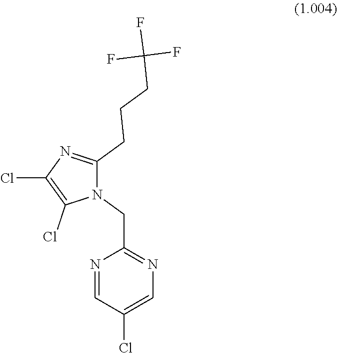 [SMILES: FC(F)(F)CCCc1nc(Cl)c(Cl)n1Cc1ncc(Cl)cn1]

To a stirred suspension of 4,5-dichloro-2-(4,4,4-trifluorobutyl)-1H-imidazole 111 (0.065 g, 0.26 mmol), KI (5 mg, 0.030 mmol) and potassium carbonate (0.09 g, 0.65 mmol) in acetonitrile (2 mL) and H₂O (0.03 mL) was added 5-chloro-2-(chloromethyl)pyrimidine hydrochloride (0.061 g, 0.29 mmol) in a single portion. The reaction was then heated to 80° C. under N₂ for 1.5 h. The reaction was cooled to room temperature and concentrated onto granulated celite. The crude product was purified by flash chromatography on silica gel using a gradient of 0-40% ethyl acetate in cyclohexane as eluent to give the desired product (70 mg, 64%) as a pale yellow oil. ¹H NMR (400 MHz, CDCl₃) 5=8.66 (s, 2H), 5.27 (s, 2H), 2.73 (t, 2H), 2.19 (tq, 2H), 2.00 (quin, 2H)

### Example 5: Preparation of 2-[[2-butyl-4-(difluoromethyl)imidazol-1-yl]methyl]-5-chloro-3-fluoro-pyridine (1.054)

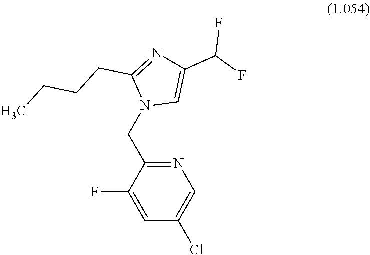 [SMILES: CCCCc1nc(C(F)F)cn1Cc1ncc(Cl)cc1F]

Step 1: Preparation of 2-butyl-4-(trifluoromethyl)-1H-imidazole (I12)

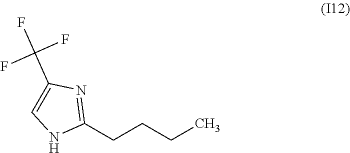

To a 250 mL flask equipped with a reflux condenser and thermometer was added sodium acetate (7.22 g, 87.07 mmol) and water (15 mL) followed by 1,1-dibromo-3,3,3-trifluoroacetone (11.87 g, 41.79 mmol) resulting a turbid solution. The reaction mixture was then heated to 100° C. for 30 minutes. The reaction was cooled to room temperature and to this solution was added pentanal (3 g, 34.83 mmol) in methanol (60 mL) slowly. Aqueous ammonia (13 g, 130 mmol) was added to the above solution and then stirred at room temperature for overnight. The residue was partitioned between ethyl acetate (200 mL) and water (200 mL) and the phases were separated. The aqueous phase was extracted with ethyl acetate (3×200 mL). The organics were combined, washed with sat. brine (100 mL) and concentrated onto granulated celite. The crude material was purified by silica gel column chromatography using 0-100% ethyl acetate/cyclohexane mixture to afford 2-butyl-4-(trifluoromethyl)-1H-imidazole 112 (6 g, 89%) as off-white solid. ¹H NMR (400 MHz, CDCl₃) δ=10.83 (br s, 1H), 7.26-7.29 (m, 1H), 2.74 (t, 2H), 1.73-1.65 (m, 2H), 1.39-1.30 (m, 2H), 0.90 (t, 3H).

Step 2: Preparation of 2-butyl-1H-imidazole-4-carbonitrile (I13)

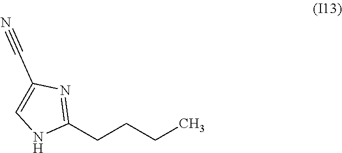

To a three necked flask was added 2-butyl-4-(trifluoromethyl)-1H-imidazole 112 (2 g, 10.40 mmol), methanol (10 mL) and aqueous ammonia (21.8 g, 156.10 mmol) and the reaction mixture was stirred at 70° C. for 6 h. After this time, the reaction mixture was cooled to room temperature and concentrated to get crude material. The crude was purified by silica gel column chromatography using 0-100% ethyl acetate/cyclohexane as eluents to afford 2-butyl-1H-imidazole-4-carbonitrile 113 (0.66 g, 42%) as off-white solid. ¹H NMR (400 MHz, METHANOL-d4) δ=7.66 (s, 1H), 2.62 (t, 2H), 1.59 (m, 2H), 1.18-1.31 (m, 2H), 0.85 (t, 3H).

Step 3: Preparation of2-butyl-1H-imidazole-4-carbaldehyde (I14)

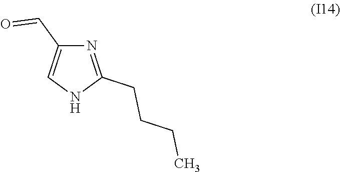

To a stirred solution of 2-butyl-1H-imidazole-4-carbonitrile 113 (0.66 g, 4.42 mmol) in dry tetrahydrofuran (13.2 mL) at −78° C. was added 1 M diisobutylaluminum hydride solution in toluene (7.6 g, 8.84 mmol) dropwise. The mixture was stirred at same temperature for 30 mins and then allowed to come to 0° C. and stirred for additional 1 hr. The reaction mixture was quenched by adding saturated NH₄Cl (15 mL) and extracted with ethyl acetate (3×50 mL). Then organic layer was washed with water (30 mL), brine (30 mL), dried on sodium sulphate, filtered, and concentrated to get crude 2-butyl-1H-imidazole-4-carbaldehyde 114 (0.45 g, 67%) as pale-yellow solid which was used directly for the next step without further purification. ¹H NMR (400 MHz, METHANOL-d4) δ=9.55 (s, 1H), 7.68 (s, 1H), 2.54-2.69 (m, 2H), 1.54-1.66 (m, 2H), 1.17-1.32 (m, 2H), 0.80-0.88 (m, 3H)

Step 4: Preparation of 2-butyl-1-[(5-chloro-3-fluoro-2-pyridyl)methyl]imidazole-4-carbaldehyde (I15)

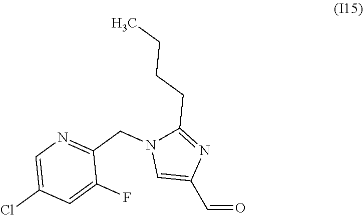 [SMILES: CCCCc1nc(C=O)cn1Cc1ncc(Cl)cc1F]

To a 25 mL flask equipped with nitrogen balloon, 2-butyl-1H-imidazole-4-carbaldehyde 114 (0.25 g, 1.64 mmol) was dissolved in acetonitrile (2.5 mL). To this, potassium carbonate (0.56 g, 4.10 mmol) and potassium iodide (0.054 g, 0.32 mmol) were added followed by 5-chloro-2-(chloromethyl)-3-fluoro-pyridine (0.35 g, 1.97 mmol). The reaction mixture was heated to 70° C. for 4 h. After this time, the reaction mixture was diluted with water (50 mL) and extracted with ethyl acetate (3×50 mL). The combined organic layer was washed with brine (30 mL), dried over sodium sulphate, filtered, and concentrated under reduced pressure to obtain the crude. This was purified by silica gel column chromatography using 0-40% ethyl acetate/cyclohexane to afford 2-butyl-1-[(5-chloro-3-fluoro-2-pyridyl)methyl]imidazole-4-carbaldehyde 115 (0.16 g, 33%) as off-white solid. ¹H NMR (400 MHz, CDC₃) 5=9.81 (s, 1H), 8.40 (d, 1H), 7.66 (s, 1H), 7.54 (dd, 1H), 5.25 (d, 2H), 2.74-2.81 (m, 2H), 1.71-1.81 (m, 2H), 1.36-1.50 (m, 2H), 0.90-0.99 (m, 3H).

Step 5: Preparation of 2-[[2-butyl-4-(difluoromethyl)imidazol-1-yl]methyl]-5-chloro-3-fluoro-pyridine (1.054)

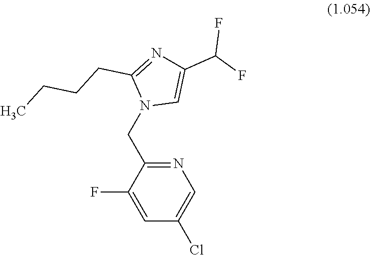 [SMILES: CCCCc1nc(C(F)F)cn1Cc1ncc(Cl)cc1F]

To a 50 mL three necked round bottomed flask with stirrer bar was added: 2-butyl-1-[(5-chloro-3-fluoro-2-pyridyl)methyl]imidazole-4-carbaldehyde 115 (128 mg, 0.43 mmol) and cooled to 0° C. To this, dichloromethane (8 mL) and diethylaminosulfur trifluoride (0.55 g, 3.46 mmol) were added at 0° C. and the mixture was stirred at room temperature for 16 h. The reaction mixture was quenched with the addition of sodium bicarbonate solution (20 mL) and extracted with ethyl acetate (3×100 mL). The combined organic layers were dried over anhydrous sodium sulphate, filtered, and evaporated to get the crude. The crude was purified by silica gel column chromatography using 0-50% ethyl acetate/cyclohexane as eluent to afford 2-[[2-butyl-4-(difluoromethyl)imidazol-1-yl]methyl]-5-chloro-3-fluoro-pyridine 1.054 (0.085 g, 58%) as pale brown liquid. ¹H NMR (400 MHz, CDC₃) δ=8.40 (d, 1H), 7.53 (dd, 1 H), 7.19 (t, 1H), 6.66 (t, 1H), 5.21 (d, 2H), 2.76-2.82 (m, 2H), 1.69-1.78 (m, 2H), 1.42 (m, 2H), 0.94 (t, 3H).

### Example 6: Preparation of [1-[(5-chloropyrimidin-2-yl)methyl]-4-(trifluoromethyl)imidazol-2-yl]methanol (1.072)

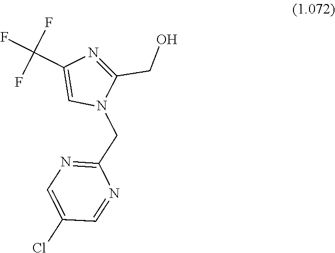 [SMILES: OCc1nc(C(F)(F)F)cn1Cc1ncc(Cl)cn1]

Step 1: Preparation of tert-butyl-dimethyl-[[4-(trifluoromethyl)-1H-imidazol-2-yl]-methoxy]-silane (I16)

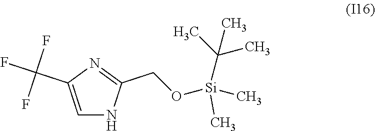 [SMILES: CC(C)(C)[Si](C)(C)OCc1nc(C(F)(F)F)c[nH]1]

To a 100 mL flask equipped with a reflux condenser and thermometer were added sodium acetate (2.38 g, 28.68 mmol), water (10 mL) and 1,1-dibromo-3,3,3-trifluoroacetone (3.91 g, 13.769 mmol). The resulting suspension was heated to 100° C. for 30 minutes. The reaction mixture was cooled to room temperature and to this solution added aqueous ammonia (8.8 g, 88 mmol) followed by 2-[tert-butyl(dimethyl)silyl]oxyacetaldehyde (2 g, 11.47 mmol) in methanol (40 mL) slowly and stirred at room temperature for 16 h. After this time, the reaction mixture was quenched by adding water (100 mL) and extracted with ethyl acetate (3×150 mL).

The organic layer was washed with brine (100 mL), dried over anhydrous sodium sulphate, filtered and concentrated to get the crude material. The crude was purified by silica gel column chromatography using 0-100% ethyl acetate/cyclohexane to afford tert-butyl-dimethyl-[[4-(trifluoromethyl)-1H-imidazol-2-yl]methoxy]silane I16 (2.3 g, 71%) as yellowish oil. ¹H NMR (400 MHz, CDCl₃): δ=9.71 (br s, 1H), 7.34 (s, 1H), 4.83 (s, 2H), 0.93 (s, 9H), 0.12 (s, 6H).

Step 2: Preparation of tert-butyl-[[1-[(5-chloropyrimidin-2-yl)methyl]-4-(trifluoromethyl)imidazol-2-yl]methoxy]-dimethyl-silane (I17)

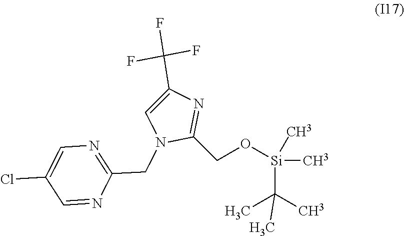 [SMILES: CC(C)(C)[Si](C)(C)OCc1nc(C(F)(F)F)cn1Cc1ncc(Cl)cn1]

To a 50 mL flask equipped with nitrogen balloon, tert-butyl-dimethyl-[[4-(trifluoromethyl)-1H-imidazol-2-yl]methoxy]silane I16 (2.3 g, 8.2 mmol) was dissolved in acetonitrile (23 mL). To this, potassium carbonate (2.8 g, 21 mmol) and potassium iodide (0.27 g, 1.6 mmol) were added, and the reaction mixture was heated to 60° C. for 16 h. After the completion of the reaction, the reaction mixture was diluted with water (150 mL) and extracted with ethyl acetate (3×100 mL). The combined organic layer was washed with brine (100 mL), dried over sodium sulphate, filtered, and concentrated under reduced pressure to obtain the crude tert-butyl-[[1-[(5-chloropyrimidin-2-yl)methyl]-4-(trifluoromethyl)imidazol-2-yl]methoxy]-dimethyl-silane 117 (4.1 g, 98%, −80% purity) which was used directly for the next step. ¹H NMR (400 MHz, CDCl₃): δ=8.65 (s, 2H), 7.34 (s, 1H), 5.49 (s, 2H), 4.81 (s, 2H), 0.81 (s, 9H), 0.0 (s, 6H).

Step 3: Preparation of [1-[(5-chloropyrimidin-2-yl)methyl]-4-(trifluoromethyl)imidazol-2-yl]methanol (1.072)

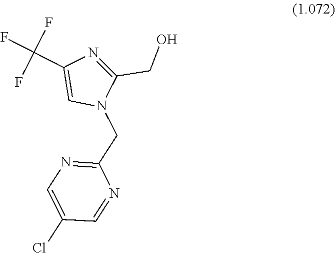 [SMILES: OCc1nc(C(F)(F)F)cn1Cc1ncc(Cl)cn1]

To a 100 mL flask was added: tert-butyl-[[1-[(5-chloropyrimidin-2-yl)methyl]-4-(trifluoromethyl)imidazol-2-yl]methoxy]-dimethyl-silane I17 (4.1 g, 5.0 mmol), ethanol (25 mL) and hydrochloric acid in deionized water (30 mL). The mixture was stirred at room temperature for 16 h. The reaction mass was quenched with water (150 mL) and extracted with ethyl acetate (3×250 ml). The combined organic layer was washed with brine solution (100 mL), dried over anhydrous sodium sulphate, filtered, and concentrated to get the crude material. This was purified by silica gel column chromatography using 0-100% ethyl acetate/cyclohexane to afford [1-[(5-chloropyrimidin-2-yl)methyl]-4-(trifluoromethyl)imidazol-2-yl]methanol 1.072 (1.0 g, 61%) as yellow liquid. ¹H NMR (400 MHz, CDCl₃) δ=8.68 (s, 2H), 7.33 (s, 1H), 5.47 (s, 2H), 4.79 (s, 2H).

### Example 7: Preparation of 5-chloro-2-[[2-(difluoromethyl)-4-(trifluoromethyl)imidazol-1-yl]methyl]pyrimidine (1.070)

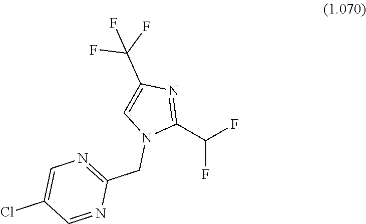 [SMILES: FC(F)c1nc(C(F)(F)F)cn1Cc1ncc(Cl)cn1]

Step 1: Preparation of 1-[(5-chloropyrimidin-2-yl)methyl]-4-(trifluoromethyl)imidazole-2-carbaldehyde (I18)

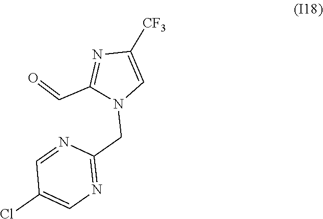 [SMILES: O=Cc1nc(C(F)(F)F)cn1Cc1ncc(Cl)cn1]

In a 3-neck flask equipped with nitrogen inlet [1-[(5-chloropyrimidin-2-yl)methyl]-4-(trifluoromethyl)imidazol-2-yl]methanol 1.072 (0.3 g, 1.02 mmol) was dissolved in acetonitrile (6 mL). The resulting reaction mass was cooled to 0° C. and (1,1-diacetyloxy-3-oxo-1A5,2-benziodoxol-1-yl) acetate (0.67 g, 1.53 mmol) was added in portion over a period of 5 minutes. The reaction mixture was stirred at room temperature for 16 h. The reaction was quenched by adding saturated solution of sodium thiosulfate (2 mL) in portion. The resulting mixture was stirred for additional 5 minutes and aqueous sodium hydrogen carbonate solution (30 mL) was added. The aqueous layer was extracted with ethyl acetate (3×50 mL). The combined organic layer was washed with brine solution (50 mL), dried over anhydrous sodium sulphate, filtered, and concentrated to get crude. This was purified by silica gel column chromatography using 0-30% ethyl acetate in cyclohexane as eluent to afford 1-[(5-chloropyrimidin-2-yl)methyl]-4-(trifluoromethyl)imidazole-2-carbaldehyde 118 (340 mg, 68%) as white solid. ¹H NMR (400 MHz, CDCl₃): δ=9.83 (d, 1H), 8.65 (s, 2H), 7.58 (s, 1H), 5.85 (s, 2H).

Step 2: Preparation of 5-chloro-2-[[2-(difluoromethyl)-4-(trifluoromethyl)imidazol-1-yl]methyl]pyrimidine (1.070)

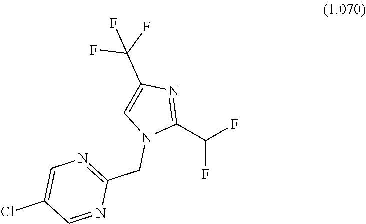 [SMILES: FC(F)c1nc(C(F)(F)F)cn1Cc1ncc(Cl)cn1]

To solution of 1-[(5-chloropyrimidin-2-yl)methyl]-4-(trifluoromethyl)imidazole-2-carbaldehyde 118 (0.34 g, 0.93 mmol) in dichloromethane (10 mL) at 0° C., diethylaminosulfur trifluoride (1.27 g, 7.48 mmol) was added dropwise. This was allowed to warm up to room temperature and stirred for 16 h. After this time, the reaction mixture was cooled to 0° C., quenched with saturated bicarbonate solution (30 mL). The layers were separated, and the aqueous layer was extracted with ethyl acetate (3×70 mL), washed with brine solution (30 mL), dried over sodium sulphate, filtered and concentrated to get the crude material. This was purified by silica gel column chromatography using 0-100% ethyl acetate in cyclohexane to afford 5-chloro-2-[[2-(difluoromethyl)-4-(trifluoromethyl)imidazol-1-yl]methyl]pyrimidine 1.070 (85 mg, 29%) as brown liquid. ¹H NMR (400 MHz, CDCl₃) δ=8.69 (s, 2H), 7.52 (s, 1H), 6.78 (t, 1H), 5.56 (s, 2H).

### Example 8: Preparation of 5-chloro-2-[[2-(chloromethyl)-4-(trifluoromethyl)imidazol-1-yl]methyl]pyrimidine (1.071)

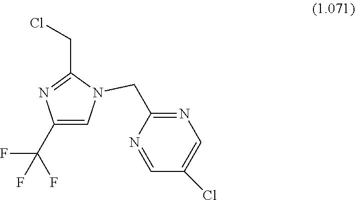 [SMILES: FC(F)(F)c1cn(Cc2ncc(Cl)cn2)c(CCl)n1]

To a solution of [1-[(5-chloropyrimidin-2-yl)methyl]-4-(trifluoromethyl)imidazol-2-yl]methanol 1.072 (0.08 g, 0.27 mmol) in acetonitrile (0.8 mL) at 0° C. under nitrogen was added thionyl chloride (0.065 g, 0.54 mmol) and stirred for 10 minutes. The reaction mixture was allowed to warm up to room temperature for 2 h. The reaction mixture was cooled to 0° C. and then quenched with sat sodium bicarbonate solution (20 mL). This was extracted with ethyl acetate (3×30 mL). The combined organic layers were dried over anhydrous sodium sulphate, filtered, and concentrated under reduced pressure to get the crude material. The crude was purified by silica gel column chromatography using 0-10% ethyl acetate in cyclohexane to afford 5-chloro-2-[[2-(chloromethyl)-4-(trifluoromethyl)imidazol-1-yl]methyl]pyrimidine 1.071 (0.053 g, 62%) as gummy mass. ⁴H NMR (400 MHz, CDCl₃) δ=8.70 (s, 2H), 7.42 (d, 1H), 5.46 (s, 2H), 4.79 (s, 2H).

### Example 9: Preparation of 1-[(5-chloropyrimidin-2-yl)methyl]-2-(tetrahydrofuran-3-ylmethyl)imidazole-4-carbonitrile (1.073)

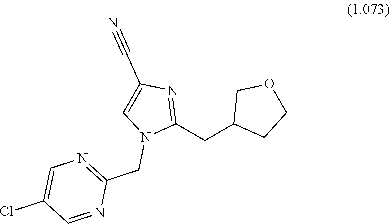 [SMILES: N#Cc1cn(Cc2ncc(Cl)cn2)c(CC2CCOC2)n1]

Step 1: Preparation of 2-tetrahydrofuran-3-ylacetaldehyde (119)

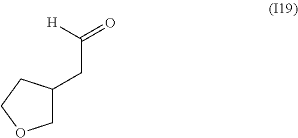

To a solution of 3-[(E)-2-methoxyvinyl]tetrahydrofuran (1.57 g, 12.2 mmol) in acetonitrile (2 mL) was added hydrogen chloride (2.5 mL, 5.0 mmol, 2 M) dropwise and the reaction mixture was stirred at room temperature for overnight. The reaction mixture was diluted with water (50 mL) and extracted with tert-butyl dimethyl ether (3×50 mL). The combined organic layer was washed with brine (50 mL), dried over sodium sulphate, filtered, and concentrated under reduced pressure to yield the crude compound. The crude compound was purified by column using 0-5% ethyl acetate in cyclohexane to afford 2-tetrahydrofuran-3-ylacetaldehyde 119 (0.9 g, 60%) as clear liquid. ¹H NMR (400 MHz, CDCl₃): δ=9.79 (s, 1H), 4.02-3.95 (m, 1H), 3.90-3.83 (m, 1H), 3.80-3.75 (s, 1H), 3.42-3.35 (m, 1H), 2.76-2.63 (m, 1H), 2.62-2.55 (m, 2H), 2.25-2.10 (m, 1H), 1.60-1.47 (m, 1H).

Step 2: Preparation of 2-(tetrahydrofuran-3-ylmethyl)-4-(trifluoromethyl)-1H-imidazole (I20)

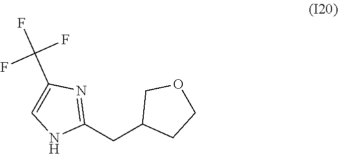 [SMILES: FC(F)(F)c1c[nH]c(CC2CCOC2)n1]

To a 100 mL flask equipped with a reflux condenser was added sodium acetate (1.63 g, 19.71 mmol), water (4.5 mL) and 1,1-dibromo-3,3,3-trifluoroacetone (2.68 g, 9.46 mmol). The reaction mixture was heated to 100° C. for 30 minutes. The reaction mixture was cooled to room temperature and to this solution added a solution of 2-tetrahydrofuran-3-ylacetaldehyde 119 (0.9 g, 7.88 mmol) in methanol (18 mL) slowly followed by aqueous ammonia (4.5 mL) dropwise and stirred at room temperature for 16 h. The reaction mixture was extracted with ethyl acetate (3×100 mL). The combined organic layer was washed with brine (100 mL), dried over anhydrous sodium sulphate, filtered, and concentrated to get the crude material. The crude product was purified by column chromatography using 0-40% ethyl acetate in cyclohexane. Upon concentration, 2-(tetrahydrofuran-3-ylmethyl)-4-(trifluoromethyl)—1H-imidazole 120 (0.7 g, 40%) was obtained as white solid. ¹H NMR (400 MHz, CDCl₃) δ=7.29-7.30 (d, 1H), 3.75-3.96 (m, 3H), 3.56 (dd, 1H), 2.84-2.88 (d, 2H), 2.76 (m, 1H), 2.12 (br dd, 1H), 1.66 (br d, 1H).

Step 3: Preparation of 2-(tetrahydrofuran-3-ylmethyl)-1H-imidazole-4-carbonitrile (I21)

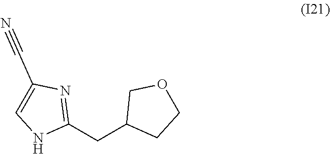 [SMILES: N#Cc1c[nH]c(CC2CCOC2)n1]

To a 15 mL vial was added 2-(tetrahydrofuran-3-ylmethyl)-4-(trifluoromethyl)-1H-imidazole 120 (370 mg, 1.68 mmol), methanol (2 mL) and aqueous ammonia (4 mL) The vial was sealed, and the reaction mixture was stirred at 70° C. for 7 h. Reaction mixture was cooled to room temperature and concentrated under reduced pressure to give the crude product. The crude product was purified by silica gel column chromatography, using 50-100% ethyl acetate and cyclohexane. Upon concentration, 2-(tetrahydrofuran-3-ylmethyl)-1H-imidazole-4-carbonitrile 121 (220 mg, 73%) was obtained as pale-yellow solid. ¹H NMR (400 MHz, MeOH-d4) b ppm 7.78 (s, 1H), 3.72-3.90 (m, 3H), 3.46 (m, 1H), 2.79 (dd, 2H), 2.61-2.71 (m, 1H), 2.05 (m, 1H), 1.60-1.68 (m, 1H).

Step 4: Preparation of 1-[(5-chloropyrimidin-2-yl)methyl]-2-(tetrahydrofuran-3-ylmethyl)imidazole-4-carbonitrile (1.073)

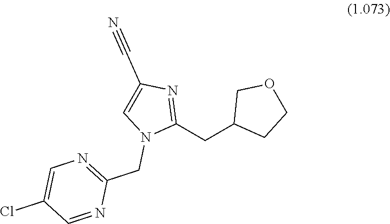 [SMILES: N#Cc1cn(Cc2ncc(Cl)cn2)c(CC2CCOC2)n1]

To a solution of 2-(tetrahydrofuran-3-ylmethyl)-1H-imidazole-4-carbonitrile 121 (100 mg, 0.56 mmol) in acetonitrile (1.2 mL) was added potassium carbonate (0.24 g, 1.69 mmol), potassium iodide (0.018 g., 0.11 mmol) followed by 5-chloro-2-(chloromethyl)pyrimidine (0.1 g, 0.62 mmol). The reaction mass was heated at 80° C. for 5 h. Reaction was cooled to room temperature, diluted with 100 ml ice cold water, extracted in ethyl acetate (3×25 ml). The organic layer was washed with brine solution (50 mL), dried over anhydrous sodium sulphate, filtered, and concentrated to get the crude material. The crude product was purified by silica gel column chromatography using 70% ethyl acetate in cyclohexane. Upon concentration 1-[(5-chloropyrimidin-2-yl)methyl]-2-(tetrahydrofuran-3-ylmethyl)imidazole-4-carbonitrile 1.073 (102 mg, 58.9%) was obtained as white solid. ¹H NMR (400 MHz, CDCl₃) b=8.67-8.70 (m, 2H), 7.54 (s, 1H), 5.28 (s, 2H), 3.85-3.94 (m, 2H), 3.76 (m, 1H), 3.47 (m, 1H), 2.73-2.85 (m, 3H), 2.15 (m, 1H), 1.60-1.65 (m, 1H).

### Example 10: Preparation of 1-[2-butyl-1-[(5-chloropyrimidin-2-yl)methyl]imidazol-4-yl]-N-methoxy-methanimine (1.076)

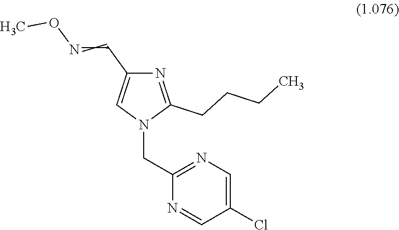 [SMILES: CCCCc1nc(C=NOC)cn1Cc1ncc(Cl)cn1]

Step 1: Preparation of 2-butyl-N,N-dimethyl-imidazole-1-sulfonamide (122)

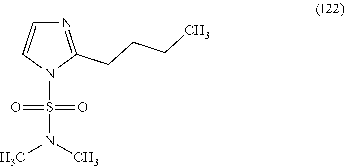

To a stirred solution of 2-butyl-1H-imidazole (800 mg, 6.44 mmol) in dimethylformamide (20 mL) at 0° C. was added sodium hydride (60% in mineral oil) (0.77 g, 19.327 mmol) and the reaction mixture was stirred for 30 min. To this solution, was added dimethylsulfamoyl chloride (1.12 g, 7.73 mmol) dropwise and stirring was continued at room temperature for 2 h. Then reaction mixture was carefully quenched by adding sat. NH₄Cl solution (−50 mL). This was extracted with ethyl acetate (3×50 mL). Then the organic layer was washed well with water (50 mL), brine (50 mL) and dried over sodium sulphate, filtered, and concentrated to get crude compound. It was then purified by silica gel column chromatography using 30-50% ethyl acetate in cyclohexane to get 2-butyl-N,N-dimethyl-imidazole-1-sulfonamide 122 (0.8 g, 53%) as clear gummy oil. ¹H NMR (400 MHz, CDC₃)═7.21 (d, 1H), 6.95 (d, 1H), 2.90-2.95 (m, 2H), 2.89 (s, 6H), 1.71-1.89 (m, 2H), 1.38-1.48 (m, 2H), 0.95 (t, 3H).

Step 2: Preparation of 2-butyl-4-formyl-N,N-dimethyl-imidazole-1-sulfonamide (I23)

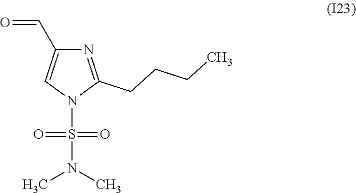 [SMILES: CCCCc1nc(C=O)cn1S(=O)(=O)N(C)C]

To a stirred solution of 2-butyl-N,N-dimethyl-imidazole-1-sulfonamide 122 (1.5 g, 6.5 mmol) in tetrahydrofuran (30 mL) at −78° C. was added n-butyl lithium (3.1 mL, 7.8 mmol, 2.5 mol/L) dropwise over 30 min. To this reaction mixture was added N,N-dimethylformamide (2.5 mL) at −78° C. and then reaction mixture was stirred at −50° C. for 30 min under nitrogen. The reaction mixture was quenched by adding −10 mL 1N hydrogen chloride carefully and the reaction mixture was stirred at room temperature for 2 h. Then the pH of the reaction mixture was adjusted to 7-8 by adding aqueous NaHCO₃ solution and then extracted with ethyl acetate (3×50 mL). The organic layer was washed well with brine (50 mL), dried on sodium sulphate, filtered, and concentrated to get crude compound. The crude was purified by silica gel column chromatography using 30-50% ethyl acetate in cyclohexane to afford 2-butyl-4-formyl-N,N-dimethyl-imidazole-1-sulfonamide 123 (1.2 g, 71%) as pale-yellow gummy mass. ¹H NMR (400 MHz, CDCl₃) 5=10.04 (s, 1H), 7.78 (s, 1H), 3.00-3.05 (m, 2H), 2.94 (s, 6H), 1.79-1.88 (m, 2H), 1.40-1.50 (m, 2H), 0.97 (t, 3H).

Step 3: Preparation of 2-butyl-1H-imidazole-4-carbaldehyde (I24)

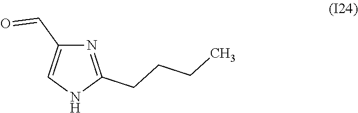

To a stirred solution of 2-butyl-4-formyl-N,N-dimethyl-imidazole-1-sulfonamide 123 (900 mg, 3.47 mmol) in methanol (0.5 mL) was added hydrochloric acid (2 mL, 36%) The mixture was stirred at 80° C. for 1 h. After this time, the reaction mixture was cooled to room temperature, neutralized by adding sat. sodium bicarbonate solution (−20 mL). Then it was extracted with ethyl acetate (3×50 mL). Then the organic layer was washed well with water (50 mL), brine (50 mL), dried on sodium sulphate, filtered, and concentrated to get 2-butyl-1H-imidazole-4-carbaldehyde 124 (0.46 g, 78%) as clear gummy mass ¹H NMR (400 MHz, CDCl₃) 5=9.66 (s, 1H), 7.75 (s, 1H), 2.81-2.85 (m, 2H), 1.69-1.83 (m, 2H), 1.31-1.46 (m, 2H), 0.89-0.97 (m, 3H).

Step 4: Preparation of 2-butyl-1-[(5-chloropyrimidin-2-yl)methyl]imidazole-4-carbaldehyde (I25)

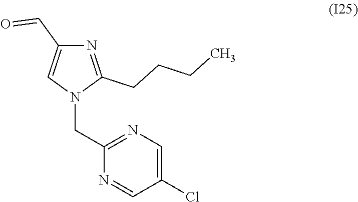 [SMILES: CCCCc1nc(C=O)cn1Cc1ncc(Cl)cn1]

To a solution of 2-butyl-1H-imidazole-4-carbaldehyde 124 (0.46 g, 3.02 mmol) in sulfolane (6.0 mL) was added potassium carbonate (1.25 g, 9.06 mmol) followed by 5-chloro-2-(chloromethyl)pyrimidine (0.60 g, 3.3 mmol). The reaction mass was heated at 60° C. for 16 h. After this time, the reaction mixture was cooled to room temperature, it was diluted with water (50 mL), extracted with ethyl acetate (3×30 mL). The organic layer was washed with brine (50 mL), dried on sodium sulphate, filtered, and concentrated to get the crude. The crude product was then purified by silica gel column chromatography using 30% ethyl acetate in cyclohexane. Upon concentration, 2-butyl-1-[(5-chloropyrimidin-2-yl)methyl]imidazole-4-carbaldehyde 125 (0.28 g, 28%) was obtained as yellowish solid. ¹H NMR (400 MHz, CDCl₃) δ=9.85 (s, 1H), 8.69 (s, 2H), 7.70 (s, 1H), 5.31 (s, 2H), 2.68-2.75 (m, 2H), 1.70-1.83 (m, 2 H), 1.34-1.45 (m, 2H), 0.93 (t, 3H).

Step 5: Preparation of 1-[2-butyl-1-[(5-chloropyrimidin-2-yl)methyl]imidazol-4-yl]-N-methoxy-methanimine (1.076)

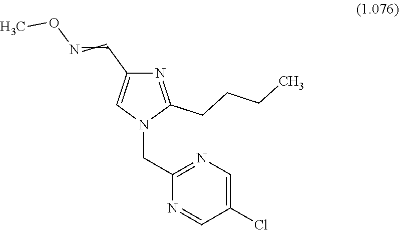 [SMILES: CCCCc1nc(C=NOC)cn1Cc1ncc(Cl)cn1]

To a stirred solution of 2-butyl-1-[(5-chloropyrimidin-2-yl)methyl]imidazole-4-carbaldehyde 125 (75 mg, 0.26 mmol) in ethanol (2 mL) was added O-methylhydroxylamine hydrochloride (76 mg, 0.81 mmol) followed by sodium acetate (0.64 g, 0.80 mmol) and the reaction mixture was heated at 50° C. for 16 h. After this time, the reaction mixture was concentrated. Then the residue was diluted with ethyl acetate (20 mL) and washed well with water (3×15 mL), brine (15 mL) dried on sodium sulphate, filtered, and concentrated to get the desired compound. Upon concentration, 1-[2-butyl-1-[(5-chloropyrimidin-2-yl)methyl]imidazol-4-yl]-N-methoxy-methanimine 1.076 (83 mg, 95%) was obtained as E/Z mixture (3.3:1) as gummy mass.

Major Isomer: ¹H NMR (400 MHz, CDCl3) δ=8.68 (s, 2H), 7.77 (s, 1H), 7.54 (s, 1 H), 5.29 (s, 2H), 4.02 (s, 3H), 2.70-2.74 (m, 2H), 1.69-1.73 (m, 2H), 1.35-1.41 (m, 2H), 0.89-0.93 (m, 3H).

Minor Isomer: ¹H NMR (400 MHz, CDCl3) 5=8.67 (s, 2H), 8.03 (s, 1H), 7.22 (s, 1H), 5.65 (s, 2H), 3.93 (s, 3H), 2.70-2.74 (m, 2H), 1.69-1.73 (m, 2H), 1.35-1.41 (m, 2 H), 0.89-0.93 (m, 3H).

### Example 11: Preparation of 2-[[2-butyl-4-(trifluoromethyl)imidazol-1-yl]methyl]-5-fluoro-pyrimidine (1.077)

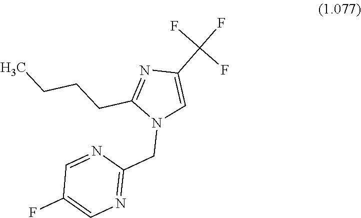 [SMILES: CCCCc1nc(C(F)(F)F)cn1Cc1ncc(F)cn1]

Step 1: Preparation of diethyl 2-(5-fluoropyrimidin-2-yl)propanedioate (I26)

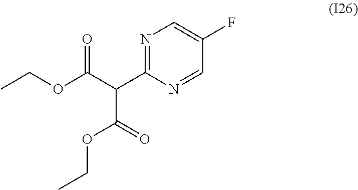 [SMILES: CCOC(=O)C(C(=O)OCC)c1ncc(F)cn1]

To a solution of 2-chloro-5-fluoro-pyrimidine (2 g, 15.09 mmol) in 1,4-dioxane (40 mL) added cesium carbonate (14.26 g, 43.76 mmol), diethyl propanedioate (4.537 mL, 29.88 mmol) and then degassed for 15 min by bubbling nitrogen. To this was added cupriooxycopper (0.432 g, 2.86 mmol), picolinic acid (0.731 g, 5.88 mmol), and heated at 130° C. for 10 h in a closed vessel. After this time, the reaction mixture was cooled to room temperature, partitioned between ethyl acetate (70 ml) and water (20 ml). Aqueous layer was re extracted with ethyl acetate (60 ml). The combined organic layer was dried over sodium sulphate, filtered, and concentrated to get the crude material. The crude material was purified by using column chromatography using 0-50% ethyl acetate in cyclohexane. Upon concentration, diethyl 2-(5-fluoropyrimidin-2-yl)propanedioate 126 (2.2 g, 40%) was obtained as yellow oil along with the decarboxylated product. ¹H NMR (400 MHz, CDCl₃): δ=8.62 (s, 2H), 5.12 (s, 1H), 4.17-4.25 (m, 4H), 1.25-1.28 (m, 6H).

Step 2: Preparation of ethyl 2-(5-fluoropyrimidin-2-yl)acetate (I27)

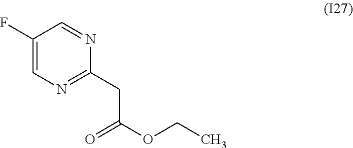

The solution of diethyl 2-(5-fluoropyrimidin-2-yl)propanedioate 126 (500 mg, 1.95 mmol) in dimethyl sulphoxide (5 mL) and the sodium chloride (0.1403 g, 2.40 mmol) and water (0.125 mL) was heated to 150° C. for 7 h. After this time, the reaction mass was quenched by adding water (100 mL) and extracted with ethyl acetate (3×200 mL). The combined organic layer was dried over sodium sulfate, filtered, and concentrate to get the crude. The crude reaction mass was purified by column chromatography by using 30% ethyl acetate in cyclohexane to afford ethyl 2-(5-fluoropyrimidin-2-yl)acetate 127 (250 mg, 69%) as yellow gummy mass. ¹H NMR (400 MHz, CDCl₃) δ=8.58 (s, 2H), 4.25 (q, 2H), 4.04 (s, 2H), 1.27 (t, 3H).

Step 3: Preparation of ethyl 2-bromo-2-(5-fluoropyrimidin-2-yl)acetate (I28)

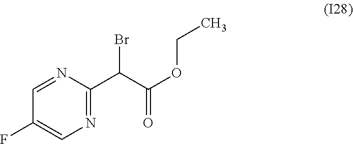

To a stirred solution of ethyl 2-(5-fluoropyrimidin-2-yl)acetate 127 (0.6 g, 3.25 mmol) in carbon tetrachloride (12 mL) was added benzoyl peroxide (0.08 g, 0.32 mmol) followed by N-bromosuccinimide (0.66 g, 3.64 mmol) and heated to reflux for 6 h. After this time, the reaction mixture was cooled to room temperature and quenched by adding water (100 mL). This was extracted with ethyl acetate (3×100 mL), dried over sodium sulfate, filtered and concentrated to get the crude product. The crude reaction mass was purified by silica gel column chromatography using ethyl 30% acetate in cyclohexane to afford ethyl 2-bromo-2-(5-fluoropyrimidin-2-yl)acetate 128 (300 mg, 35%) as yellow oil. ¹H NMR (400 MHz, CDCl₃) δ=8.63 (s, 2H), 5.69 (s, 1 H), 4.30 (q, 2H), 1.31 (t, 3H).

Step 4: Preparation of ethyl 2-[2-butyl-4-(trifluoromethyl)imidazol-1-yl]-2-(5-fluoropyrimidin-2-yl)acetate (I29)

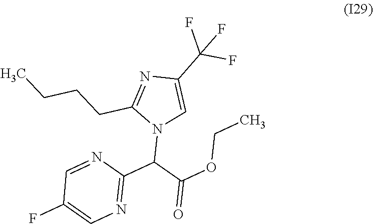 [SMILES: CCCCc1nc(C(F)(F)F)cn1C(C(=O)OCC)c1ncc(F)cn1]

To a solution of 2-bromo-2-(5-fluoropyrimidin-2-yl)acetate 128 (300 mg, 1.14 mmol) in sulfolane (6 mL) were added potassium carbonate (0.78 g, 5.70 mmol) followed by 2-butyl-4-(trifluoromethyl)-1H-imidazole 112 (0.21 g, 1.1404 mmol). The mixture was then heated to 120° C. for 1 h. After this time, the reaction mass was quenched by adding sat. solution of ammonium chloride (30 mL) and extracted with ethyl acetate (3×50 mL), dried over sodium sulfate, filtered, and concentrated. The crude product was purified by silica gel column chromatography using 20% ethyl acetate in cyclohexane to afford ethyl 2-[2-butyl-4-(trifluoromethyl)imidazol-1-yl]-2-(5-fluoropyrimidin-2-yl)acetate 129 (90 mg, 21%) as colorless oil. ¹H NMR (400 MHz, CDCl₃) δ=8.64 (s, 2H), 7.52-7.59 (m, 1H), 6.19 (s, 1H), 4.31 (m, 2H), 2.80 (t, 2 H), 1.70-1.79 (m, 2H), 1.37-1.45 (m, 2H), 1.27 (t, 3H), 0.90-0.97 (m, 3H).

Step 5: Preparation of 2-[[2-butyl-4-(trifluoromethyl)imidazol-1-yl]methyl]-5-fluoro-pyrimidine (1.077)

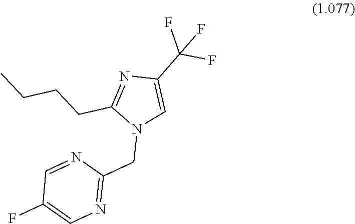 [SMILES: CCCCc1nc(C(F)(F)F)cn1Cc1ncc(F)cn1]

To a solution of ethyl 2-[2-butyl-4-(trifluoromethyl)imidazol-1-yl]-2-(5-fluoropyrimidin-2-yl)acetate 129 (50 mg, 0.13 mmol) in methanol (1 mL) was added 2 M sodium hydroxide (1.336 mL) and the reaction mixture was stirred at room temperature overnight. The reaction mass was quenched by adding 2N hydrochloric acid (10 mL) and diluted with water (30 mL). The aqueous layer was extracted with ethyl acetate (3×50 mL) and dried over sodium sulfate, filtered and concentrated. The residue was purified by silica gel column chromatography using 30% ethyl acetate in cyclohexane to afford 2-[[2-butyl-4-(trifluoromethyl)imidazol-1-yl]methyl]-5-fluoro-pyrimidine 1.077 (12 mg, 29%) as yellow oil. ¹H NMR (400 MHz, CDCl₃) δ=8.59 (s, 2H), 7.31 (d, 1H), 5.28 (s, 2H), 2.69-2.75 (m, 2H), 1.71-1.78 (m, 2H), 1.38 (br d, 2H), 0.91 (t, 3H).

### Example 12: Preparation of 2-[[2-butyl-4-(difluoromethyl)imidazol-1-yl]methyl]-5-chloro-pyrimidine (1.053)

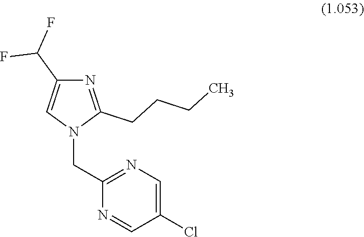 [SMILES: CCCCc1nc(C(F)F)cn1Cc1ncc(Cl)cn1]

To a stirred solution of 2-butyl-1-[(5-chloropyrimidin-2-yl)methyl]imidazole-4-carbaldehyde 125 (100 mg, 0.3588 mmol) in dichloromethane (5 mL) at 0° C. was added diethylaminosulfur trifluoride (0.46 g, 2.870 mmol) dropwise and allowed to stir at room temperature for overnight. After this time, the reaction mixture was carefully quenched by adding saturated sodium bicarbonate solution (−20 mL). Then it was extracted with dichloromethane (3×30 mL). The combined organic layer was washed well with water (30 mL), brine (30 mL) and dried over sodium sulphate. It was concentrated to get the crude material. It was then purified by silica gel column chromatography using 0-30% ethyl acetate in cyclohexane to get 2-[[2-butyl-4-(difluoromethyl)imidazol-1-yl]methyl]-5-chloro-pyrimidine 1.053 (55 mg, 51%) as gummy oil. ¹H NMR (400 MHz, CDCl3) δ=8.68 (s, 2H), 7.21 (t, 1H), 6.67 (t, 1H), 5.26 (s, 2H), 2.68-2.74 (m, 2H), 1.65-1.79 (m, 2H), 1.34-1.44 (m, 2H), 0.92 (t, 3H).

### Example 13: Preparation of 3-[1-[(5-chloropyrimidin-2-yl)methyl]-4-(trifluoromethyl)imidazol-2-yl]-N-methoxy-propan-1-imine (1.051)

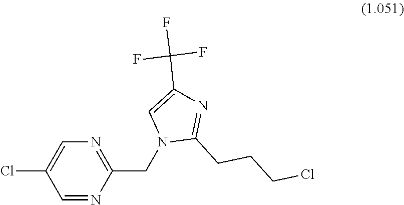 [SMILES: FC(F)(F)c1cn(Cc2ncc(Cl)cn2)c(CCCCl)n1]

Step 1: Preparation of 2-but-3-enyl-4-(trifluoromethyl)-1H-imidazole (I30)

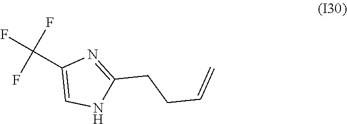

To a 100 mL flask equipped with a reflux condenser and thermometer were added sodium acetate (7.4 g, 89 mmol), water (15 mL) and 1,1-dibromo-3,3,3-trifluoroacetone (12 g, 43 mmol) resulting a turbid solution/suspension. The reaction mixture was heated to 100° C. for 30 minutes. After this time, the reaction mixture was cooled to room temperature. To this solution were added 4-pentenal (3.0 g, 36 mmol) in methanol (60 mL) followed by aqueous ammonia (15 mL, 130 mmol) slowly and stirred at room temperature for 16 h. The reaction was extracted with ethyl acetate (3×200 mL). The combined organic layer was washed saturated bicarbonate solution (100 ml) and then with brine (100 mL), dried over anhydrous sodium sulphate, filtered, and concentrated to get the crude material. The crude was washed with tert-butyl dimethyl ether and dried to get 2-but-3-enyl-4-(trifluoromethyl)—1H-imidazole 130 (6.4 g, 94%) as yellow solid. ¹H NMR (400 MHz, DMSO-d6) δ=12.37 (br s, 1H) 7.61 (s, 1H), 5.83 (m, 1H), 4.95-5.08 (m, 2H), 2.69-2.75 (m, 2 H), 2.34-2.48 (m, 2H).

Step 2: Preparation of 2-[[2-but-3-enyl-4-(trifluoromethyl)imidazol-1-yl]methyl]-5-chloro-pyrimidine (I31)

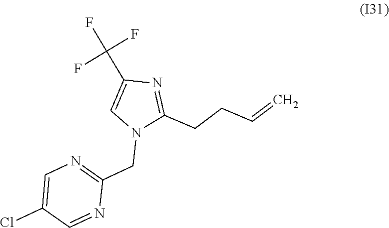 [SMILES: C=CCCc1nc(C(F)(F)F)cn1Cc1ncc(Cl)cn1]

To a solution of 2-but-3-enyl-4-(trifluoromethyl)-1H-imidazole 130 (6.4 g, 34 mmol) in sulfolane (56 mL) was added potassium carbonate (14 g, 100 mmol) followed by 5-chloro-2-(chloromethyl)pyrimidine (6.7 g, 37 mmol). The reaction mixture was heated at 100° C. for 6 h. After this time, the reaction mixture was cooled to room temperature, diluted with 100 ml ice cold water, extracted with ethyl acetate (4×500 mL). The combined organic layer was washed with brine solution (200 mL), dried over anhydrous sodium sulphate, filtered, and concentrated to get the crude material. This was purified by silica gel column chromatography using 30% ethyl acetate in cyclohexane to afford 2-[[2-but-3-enyl-4-(trifluoromethyl)imidazol-1-yl]methyl]-5-chloro-pyrimidine 131 (7.3 g, 68%) as dark yellow oil. ¹H NMR (400 MHz, CDCl₃): δ=8.68 (s, 2H), 7.31 (d, 1H), 5.84 (m, 1H), 5.26 (s, 2H), 4.96-5.08 (m, 2H), 2.77-2.83 (m, 2H), 2.47-2.55 (m, 2H)

Step 3: Preparation of 4-[1-[(5-chloropyrimidin-2-yl)methyl]-4-(trifluoromethyl)imidazol-2-yl]butane-1,2-diol (I32)

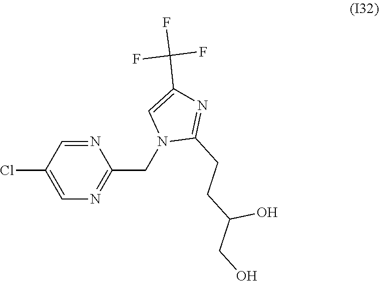 [SMILES: OCC(O)CCc1nc(C(F)(F)F)cn1Cc1ncc(Cl)cn1]

In a 100 ml flask equipped with nitrogen inlet, 2-[[2-but-3-enyl-4-(trifluoromethyl)imidazol-1-yl]methyl]-5-chloro-pyrimidine 131 (6 g, 18.95 mmol) was dissolved in tetrahydrofuran (120 mL) and water (60 mL). To this were added potassium osmate(VI) dihydrate (0.1388 g, 0.3789 mmol) followed by 4-methyl-4-oxido-morpholin-4-ium (4.53 g, 37.89 mmol) at room temperature. After addition, the reaction mixture was stirred at room temperature overnight. Reaction mixture was quenched with 10% sodium thiosulphate solution (complete quenching of osmium tetraoxide was checked by starch paper) and was extracted with 10% methanol in ethyl acetate (6×100 ml). The organic layer was washed with brine solution (100 ml), dried over sodium sulphate, filtered, and concentrated to get 4-[1-[(5-chloropyrimidin-2-yl)methyl]-4-(trifluoromethyl)imidazol-2-yl]butane-1,2-diol 132 (6.6 g, 99%) as light brown gummy mass. ¹H NMR (400 MHz, CDCl₃) δ=8.68 (s, 2H), 7.28-7.31 (m, 1 H), 5.29 (s, 2H), 3.64-3.76 (m, 1H), 3.58 (dd, 1H), 3.46 (dd, 1H), 2.89 (t, 2H), 1.82-2.00 (m, 2H).

Step 4: Preparation of 3-[1-[(5-chloropyrimidin-2-yl)methyl]-4-(trifluoromethyl)imidazol-2-yl]propanal (133)

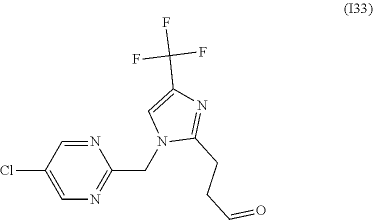 [SMILES: O=CCCc1nc(C(F)(F)F)cn1Cc1ncc(Cl)cn1]

In a 50 ml flask equipped with nitrogen inlet, 4-[1-[(5-chloropyrimidin-2-yl)methyl]-4-(trifluoromethyl)imidazol-2-yl]butane-1,2-diol 132 (6.3 g, 18 mmol) was dissolved in tetrahydrofuran (130 mL) and water (63 mL). To this solution was added sodium periodate (7.7 g, 36 mmol) at room temperature and then reaction mass was stirred at room temperature for 2 h. To the reaction mixture, 10% solution of sodium thiosulphate was added and stirred for 15 minutes. To this ethyl acetate (50 mL) and water (50 mL) were added. The aqueous layer was re-extracted with ethyl acetate (2×100 ml) and the obtained organic layer was dried over anhydrous sodium sulphate, filtered, and concentrated. The crude product was purified by silica gel column chromatography using 20% ethyl acetate in cyclohexane to afford 3-[1-[(5-chloropyrimidin-2-yl)methyl]-4-(trifluoromethyl)imidazol-2-yl]propanal 133 (4.8 g, 84%). ¹H NMR (400 MHz, CDCl₃) 5=9.83 (s, 1H), 8.67 (s, 2H), 7.26-7.30 (m, 1H), 5.34 (s, 2H), 3.09-3.14 (m, 2H), 2.96-3.01 (m, 2H).

Step 5: Preparation of 3-[1-[(5-chloropyrimidin-2-yl)methyl]-4-(trifluoromethyl)imidazol-2-y]propan-1-ol (I34)

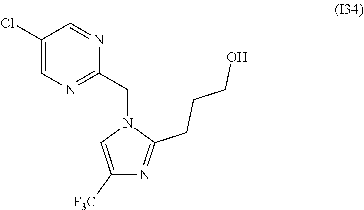 [SMILES: OCCCc1nc(C(F)(F)F)cn1Cc1ncc(Cl)cn1]

To a 2-neck flask, equipped with a nitrogen inlet, was added 3-[1-[(5-chloropyrimidin-2-yl)methyl]-4-(trifluoromethyl)imidazol-2-yl]propanal 133 (0.5 g, 1.56 mmol) in tetrahydrofuran (10 mL). This was cooled to <5° C. and added sodium borohydride (0.030 g, 0.78 mmol) in portions. The reaction mass was stirred at room temperature for 16 h. After this time, the reaction mixture was quenched with ammonium chloride (30 mL), stirred for 15 min. It was extracted with ethyl acetate (3×100 ml). Combined organic layer was washed with brine solution (50 mL), dried over sodium sulphate, filtered, and concentrated. The residue was purified by silica gel column chromatography using 40% ethyl acetate in cyclohexane. Upon concentration, 3-[1-[(5-chloropyrimidin-2-yl)methyl]-4-(trifluoromethyl)imidazol-2-yl]propan-1-ol 134 (0.31 g, 60%) was obtained as gummy mass. ¹H NMR (400 MHz, CDCl₃) δ=8.68 (s, 2H), 7.29 (s, 1H), 5.29 (s, 2H), 3.65-3.72 (m, 2H), 2.86-2.91 (m, 2H), 1.99-2.03 (m, 2 H).

Step 6: Preparation of 5-chloro-2-[[2-(3-chloropropyl)-4-(trifluoromethyl)imidazol-1-yl]methyl]pyrimidine (1.051)

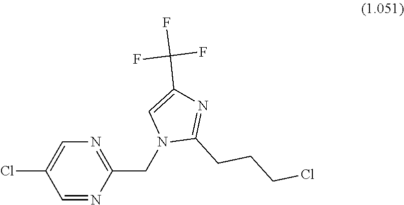 [SMILES: FC(F)(F)c1cn(Cc2ncc(Cl)cn2)c(CCCCl)n1]

To a stirred solution of 3-[1-[(5-chloropyrimidin-2-yl)methyl]-4-(trifluoromethyl)imidazol-2-yl]propan-1-ol 134 (0.135 g, 0.42 mmol) in acetonitrile (2.7 mL) at 0° C. was added thionyl chloride (0.100 g, 0.84 mmol). The reaction mixture was allowed to warm up to room temperature and stirred for 16. After this time, the reaction mixture was slowly poured into ice cold solution of sodium bicarbonate (30 mL). The reaction mass was extracted with ethyl acetate (3×100 ml). The combined organic layer was washed with brine solution (50 mL), dried over sodium sulphate, filtered, and concentrated to get the crude material. The crude product was purified by column chromatography using 20% ethyl acetate in cyclohexane to afford 5-chloro-2-[[2-(3-chloropropyl)-4-(trifluoromethyl)imidazol-1-yl]methyl]pyrimidine 1.051 (0.12 g, 84%). ¹H NMR (400 MHz, CDCl₃) 5=8.68 (s, 2H), 7.30 (d, 1H), 5.30 (s, 2 H), 3.63 (t, 2H), 2.90 (t, 2H), 2.25-2.33 (m, 2H).

### Example 14: Preparation of 3-[1-[(5-chloropyrimidin-2-yl)methyl]-4-(trifluoromethyl)-imidazol-2-yl]-N-methoxy-propan-1-imine (1.007)

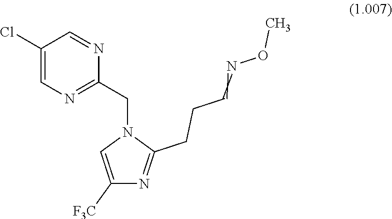 [SMILES: CON=CCCc1nc(C(F)(F)F)cn1Cc1ncc(Cl)cn1]

To a clean dry 50 mL flask equipped with nitrogen balloon, 3-[1-[(5-chloropyrimidin-2-yl)methyl]-4-(trifluoromethyl)imidazol-2-yl]propanal 133 (0.2 g, 0.62 mmol) was dissolved in acetonitrile (4 mL). To this solution was added N-methoxyammonium chloride (0.104 g, 1.25 mmol) followed by N,N-diethylethanamine (0.16 g, 1.56 mmol). The reaction mixture was stirred at room temperature for 16h. Reaction mixture was quenched with water (15 mL) and was extracted with ethyl acetate (3×80 ml). The organic layer washed with brine solution, dried over sodium sulphate, filtered, and concentrated to get the crude material. The crude was purified by silica gel column chromatography using ethyl acetate and cyclohexane as eluent. Desired product was collected by eluting with 30% ethyl acetate in cyclohexane. Upon concentration, 3-[1-[(5-chloropyrimidin-2-yl)methyl]-4-(trifluoromethyl)-imidazol-2-yl]-N-methoxy-propan-1-imine 1.007 (0.175 g, 80%) was obtained as off-white solid as E/Z mixture (2:3)

Major isomer: ¹H NMR (400 MHz, CDCl₃) b ppm 8.69 (s, 2H), 7.45 (t, 1H), 7.31 (dd, 2H), 5.31 (s, 2H), 3.80 (s, 3H), 2.88-2.96 (m, s H), 2.70-2.79 (m, s H). Minor isomer: ¹H NMR (400 MHz, CDCl₃) δ=8.69 (s, 2H), 7.32 (dd, 2H), 6.77 (t, 1 H), 5.28 (s, 2H), 3.85 (s, 3H), 2.88-2.96 (m, s H), 2.70-2.79 (m, s H).

### Example 15: Preparation of 5-chloro-2-[[2-(3,3-difluoropropyl)-4-(trifluoromethyl)-imidazol-1-yl]methyl]-pyrimidine (1.010)

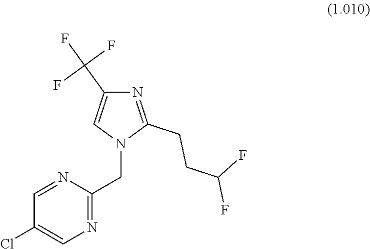 [SMILES: FC(F)CCc1nc(C(F)(F)F)cn1Cc1ncc(Cl)cn1]

To a solution of 3-[1-[(5-chloropyrimidin-2-yl)methyl]-4-(trifluoromethyl)imidazol-2-yl]propanal 133 (0.2 g, 0.62 mmol) in toluene (2.09 mL) at 0° C. was added N-ethyl-N-(trifluoro-λ⁴-sulfanyl)ethanamine (0.26 g, 1.56 mmol) dropwise. This was allowed to attain room temperature and stirred at overnight. The reaction was cooled to 0° C., quenched with saturated bicarbonate solution (30 mL). The layers were separated.

The organic layer was extracted with ethyl acetate (3×50 ml), washed with brine solution (30 mL), water (30 mL), dried over sodium sulphate, filtered, and concentrated to get the crude material. The crude product was purified by silica gel column chromatography using ethyl acetate and cyclohexane. Desired product was eluted with 20% ethyl acetate in cyclohexane. Upon concentration, 5-chloro-2-[[2-(3,3-difluoropropyl)-4-(trifluoromethyl)imidazol-1-yl]methyl]pyrimidine 1.010 (0.066 g, 31%) was isolated as off white solid. ¹H NMR (400 MHz, CDCl₃) 5=8.68 (s, 2H), 7.31 (s, 1H), 5.99 (tt, 1H), 5.26 (s, 2H), 2.88 (t, 2H), 2.32-2.47 (m, 2H).

TABLE 1
Compounds of the present invention

| | | ¹HNMR (400 MHz, CDCl₃ unless |
|---|---|---|
| COMPOUND | STRUCTURE | otherwise stated) |
| 1.001 | 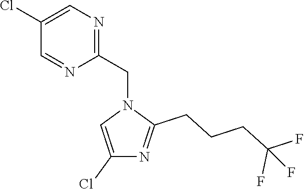 [SMILES: FC(F)(F)CCCc1nc(Cl)cn1Cc1ncc(Cl)cn1] | 8.59 (s, 2 H), 6.77 (s, 1 H), 5.10 (s, 2 H), 2.69 (t, 2 H), 2.19-2.03 (m, 2 H), 2.00-1.89 (m, 2 H) |
| 1.002 | 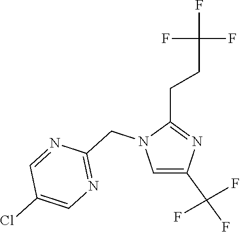 [SMILES: FC(F)(F)CCc1nc(C(F)(F)F)cn1Cc1ncc(Cl)cn1] | 8.69 (s, 2 H), 7.34 − 7.31 (m, 1 H), 5.27 (s, 2 H), 3.04 − 2.94 (m, 2 H), 2.82 − 2.64 (m, 2 H) |
| 1.003 | 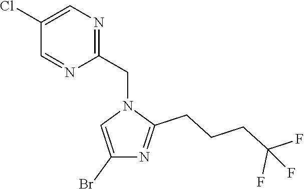 [SMILES: FC(F)(F)CCCc1nc(Br)cn1Cc1ncc(Cl)cn1] | 8.67 (s, 2 H), 6.93 (s, 1 H), 5.20 (s, 2 H), 2.78 (t, 2 H), 2.29 − 2.10 (m, 2 H), 2.09 − 1.97 (m, 2 H) |
| 1.004 | 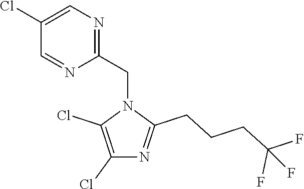 [SMILES: FC(F)(F)CCCc1nc(Cl)c(Cl)n1Cc1ncc(Cl)cn1] | 8.66 (s, 2 H), 5.27 (s, 2 H), 2.73 (t, 2 H), 2.19 (tq, 2 H), 2.00 (quin, 2 H) |
| 1.005 | 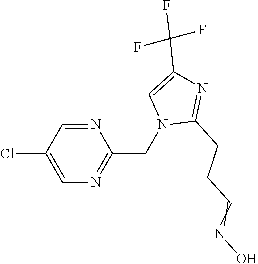 [SMILES: ON=CCCc1nc(C(F)(F)F)cn1Cc1ncc(Cl)cn1] | 3:2 mixture of isomer 1.005-1 and 1.005-2 respectively. Isomer 1.005-1: 8.69 (s, 2 H) 7.52 (t, 1 H) 7.29 − 7.34 (m, 1 H) 5.30 (s, 2 H) 2.91 − 3.01 (m, 2 H) 2.79 − 2.88 (m, 1 H) 2.69 − 2.79 (m, 1 H) Isomer 1.005-2: 8.69 (s, 2 H) 7.29 − 7.34 (m, 1 H) 6.87 (t, 1 H), 5.30 (s, 2 H) 2.91 − 3.01 (m, 2 H) 2.79 − 2.88 (m, 1 H) 2.69 − 2.79 (m, 1 H) |
| 1.006 | 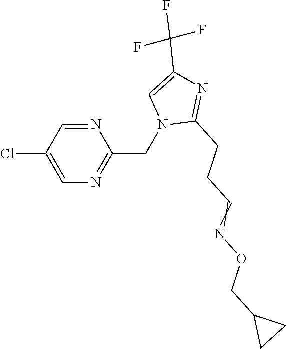 [SMILES: FC(F)(F)c1cn(Cc2ncc(Cl)cn2)c(CCC=NOCC2CC2)n1] | 3:2 mixture of isomer 1.006-1 and isomer 1.006-2 respectively. Isomer 1.006-1: 8.68 (s, 2 H), 7.50 (t, 1 H), 7.28 − 7.31 (m, 1 H), 5.30 (s, 2 H), 3.80 (d, 2 H), 2.90 − 2.96 (m, 2 H), 2.70 − 2.77 (m, 2 H), 1.05 − 1.15 (m, 1 H), 0.49 − 0.57 (m, 2 H), 0.21 − 0.28 (m, 2 H). Isomer 1.006-2: 8.68 (s, 2 H), 7.28 − 7.31 (m, 1 H), 6.77 (t, 1 H), 5.30 (s, 2 H), 3.86 (d, 2 H), 2.90 − 2.96 (m, 2 H), 2.77 − 2.85 (m, 2 H), 1.05 − 1.15 (m, 1 H), 0.49 − 0.57 (m, 2 H), 0.21 − 0.28 (m, 2 H). |
| 1.007 | 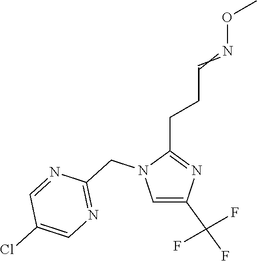 [SMILES: CON=CCCc1nc(C(F)(F)F)cn1Cc1ncc(Cl)cn1] | 3:2 mixture of isomer 1.007-1 and isomer 1.007-2 respectively Isomer 1.007-1: 8.69 (s, 2 H), 7.45 (t, 1 H), 7.31 (dd, 1 H), 5.31 (s, 2 H), 3.80 (s, 3 H), 2.88 − 2.96 (m, 2 H), 2.70 − 2.79 (m, 2 H), Isomer 1.007-2: 8.69 (s, 2 H), 7.32 (dd, 1 H), 6.77 (t, 1 H), 5.28 (s, 2 H), 3.85 (s, 3 H), 2.88 − 2.96 (m, 2 H), 2.70 − 2.79 (m, 2 H) |
| 1.008 | 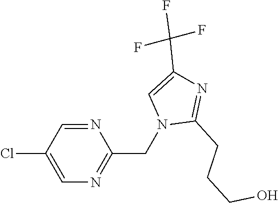 [SMILES: OCCCc1nc(C(F)(F)F)cn1Cc1ncc(Cl)cn1] | 8.68 (s, 2 H), 7.30 (s, 1 H), 5.29 (s, 2 H), 3.69 (t, 2 H), 2.88 (t, 2 H), 1.91 − 2.12 (m, 2 H) |
| 1.009 | 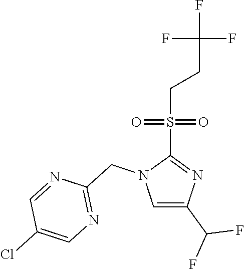 [SMILES: O=S(=O)(CCC(F)(F)F)c1nc(C(F)F)cn1Cc1ncc(Cl)cn1] | 8.66 (s, 2 H), 7.40 (t, 1 H), 6.69 (t, 1 H), 5.81 (s, 2 H), 3.76 − 3.68 (m, 2 H), 2.84 − 2.64 (m, 2 H) |
| 1.010 | 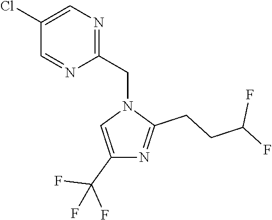 [SMILES: FC(F)CCc1nc(C(F)(F)F)cn1Cc1ncc(Cl)cn1] | 8.68 (s, 2 H), 7.31 (s, 1 H), 5.99 (tt, 1 H), 5.26 (s, 2 H), 2.88 (t, 2 H), 2.32 − 2.47 (m, 2 H) |
| 1.011 | 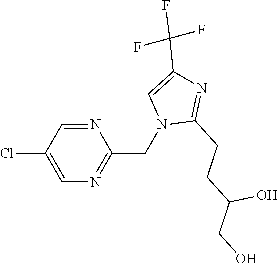 [SMILES: OCC(O)CCc1nc(C(F)(F)F)cn1Cc1ncc(Cl)cn1] | 8.68 (s, 2 H), 7.28 − 7.31 (m, 1 H), 5.29 (s, 2 H), 3.64 − 3.76 (m, 1 H), 3.58 (dd, 1 H), 3.46 (dd, 1 H), 2.89 (t, 2 H), 1.82 − 2.00 (m, 2 H) |
| 1.012 | 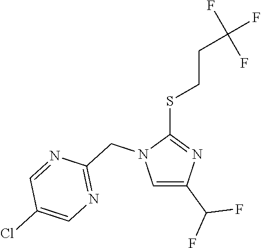 [SMILES: FC(F)c1cn(Cc2ncc(Cl)cn2)c(SCCC(F)(F)F)n1] | 8.67 (s, 2 H), 7.32 (t, 1 H), 6.65 (t, 1 H), 5.38 (s, 2 H), 3.23 − 3.14 (m, 2 H), 2.62 − 2.48 (m, 2 H) |
| 1.013 | 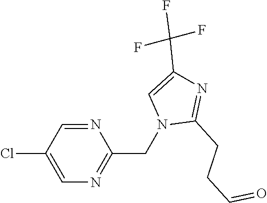 [SMILES: O=CCCc1nc(C(F)(F)F)cn1Cc1ncc(Cl)cn1] | 9.83 (s, 1 H) 8.67 (s, 2 H) 7.29 (d, 1 H) 5.34 (s, 2 H) 3.10 (d, 2 H) 2.99 (d, 2 H) |
| 1.015 | 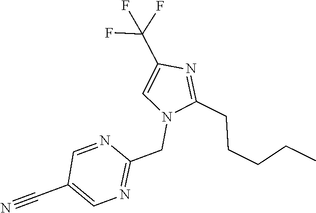 [SMILES: CCCCCc1nc(C(F)(F)F)cn1Cc1ncc(C#N)cn1] | 8.88 (s, 1 H), 8.00 (d, 1 H), 7.24 (s, 1 H), 7.06 (d, 1 H), 5.28 (s, 2 H), 2.62 (t, 2 H), 1.73-1.62 (m, 2 H), 1.35-1.21 (m, 4H), 0.85 (t, 3H) |
| 1.016 | 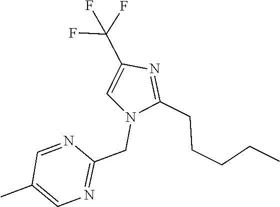 [SMILES: CCCCCc1nc(C(F)(F)F)cn1Cc1ncc(C)cn1] | 8.52 (s, 2 H), 7.31 (s, 1 H), 5.21 (s, 2 H), 2.70 (t, 2 H), 2.32 (s, 3 H), 1.75-1.62 (m, 2 H), 1.36-1.21 (m, 4 H), 0.90-0.81 (m, 3 H) |
| 1.017 | 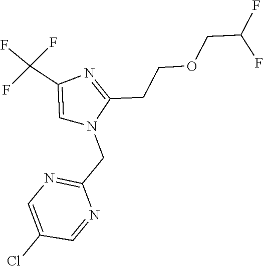 [SMILES: FC(F)COCCc1nc(C(F)(F)F)cn1Cc1ncc(Cl)cn1] | 8.67 (s, 2 H), 7.31 (s, 1 H), 5.79 (tt, 1 H), 5.35 (s, 2 H), 3.93 (t, 2 H), 3.64 (dt, 2 H), 3.06 (t, 2 H) |
| 1.018 | 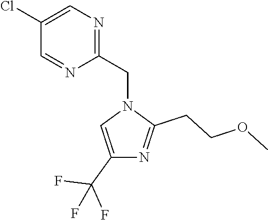 [SMILES: COCCc1nc(C(F)(F)F)cn1Cc1ncc(Cl)cn1] | 8.67 (s, 2 H), 7.31 (s, 1 H), 5.38 (s, 2 H), 3.76 − 3.65 (m, 2 H), 3.27 (s, 3 H), 3.02 (t, 2 H) |
| 1.019 | 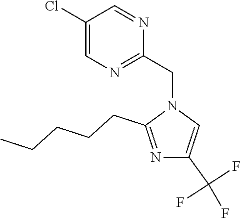 [SMILES: CCCCCc1nc(C(F)(F)F)cn1Cc1ncc(Cl)cn1] | 8.68 (s, 2 H), 7.30 (s, 1 H), 5.25 (s, 2 H), 2.72-2.65 (m, 2 H), 1.78-1.64 (m, 2 H), 1.38-1.22 (m, 4 H), 0.91-0.81 (m, 3H) |
| 1.020 | 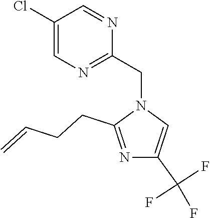 [SMILES: C=CCCc1nc(C(F)(F)F)cn1Cc1ncc(Cl)cn1] | 8.68 (s, 2 H), 7.30 (q, 1 H), 5.84 (tdd, 1 H), 5.26 (s, 2 H), 5.05 (qd, 1 H), 4.98 (qd, 1 H), 2.80 (m, 2 H), 2.51 (m, 2 H) |
| 1.021 | 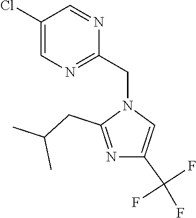 [SMILES: CC(C)Cc1nc(C(F)(F)F)cn1Cc1ncc(Cl)cn1] | 8.69 (s, 2 H), 7.31 (q, 1 H), 5.27 (s, 2 H), 2.60 (d, 2 H), 2.13 (quind, 1 H), 0.94 (d, 6 H) |
| 1.022 | 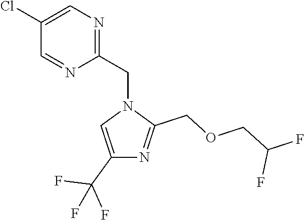 [SMILES: FC(F)COCc1nc(C(F)(F)F)cn1Cc1ncc(Cl)cn1] | 8.67 (s, 2 H), 7.41 (d, 1 H), 5.76 (tt, 1 H), 5.44 (s, 2 H), 4.78 (s, 2 H), 3.66 (dt, 2 H) |
| 1.023 | 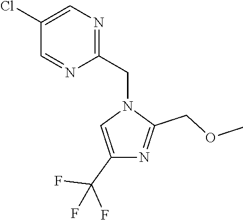 [SMILES: COCc1nc(C(F)(F)F)cn1Cc1ncc(Cl)cn1] | 8.67 (s, 2 H), 7.39 (m, 1 H), 5.45 (s, 2 H), 4.61 (s, 2 H), 3.29 (s, 3 H) |
| 1.025 | 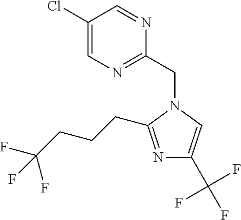 [SMILES: FC(F)(F)CCCc1nc(C(F)(F)F)cn1Cc1ncc(Cl)cn1] | 8.68 (s, 2 H), 7.32 (d, 1 H), 5.26 (s, 2 H), 2.82 (t, 2 H), 2.20 (m, 2 H), 2.05 (m, 2 H) |
| 1.026 | 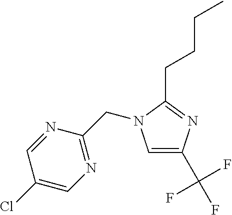 [SMILES: CCCCc1nc(C(F)(F)F)cn1Cc1ncc(Cl)cn1] | 8.68 (s, 2 H), 7.31 − 7.28 (m, 1 H), 5.25 (s, 2 H), 2.74 − 2.68 (m, 2 H), 1.76 − 1.66 (m, 2 H), 1.44 − 1.32 (m, 2 H), 0.91 (t, 3 H) |
| 1.027 | 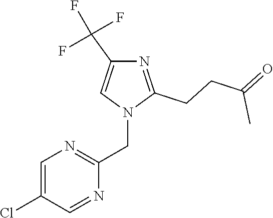 [SMILES: CC(=O)CCc1nc(C(F)(F)F)cn1Cc1ncc(Cl)cn1] | 8.68 (s, 2 H), 7.25 − 7.31 (m, 1 H), 5.36 (s, 2 H), 3.09 (t, 2 H), 2.93 (t, 2 H), 2.12 (s, 3 H). |
| 1.028 | 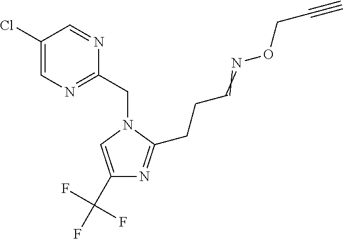 [SMILES: C#CCON=CCCc1nc(C(F)(F)F)cn1Cc1ncc(Cl)cn1] | 3:2 mixture of isomers 1.028-1 and 1.028-2 respectively. Isomer 1.028-1: 8.68 (s, 2 H), 7.52 (t, 1 H), 7.30 (dd, 1 H), 5.32 (s, 2 H), 4.58 (d, 2 H), 2.90 − 2.97 (m, 2 H), 2.77 − 2.84 (m, 2 H), 2.45 (t, 1 H) Isomer 1.028-2: 8.68 (s, 2 H), 7.29 (dd, 1 H), 6.89 (t, 1 H), 5.28 (s, 2 H), 4.64 (d, 2 H), 2.90 − 2.97 (m, 2 H) 2.77 − 2.84 (m, 2 H) 2.45 (t, 1 H) |
| 1.029 | 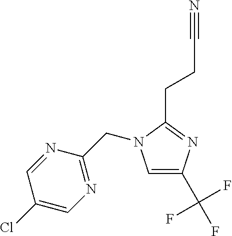 [SMILES: N#CCCc1nc(C(F)(F)F)cn1Cc1ncc(Cl)cn1] | 8.69 (s, 2 H), 7.34 (s, 1 H), 5.29 (s, 2 H), 3.10 − 3.15 (m, 2 H), 2.92 − 2.99 (m, 2 H) |
| 1.031 | 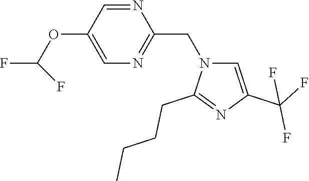 [SMILES: CCCCc1nc(C(F)(F)F)cn1Cc1ncc(OC(F)F)cn1] | |
| 1.032 | 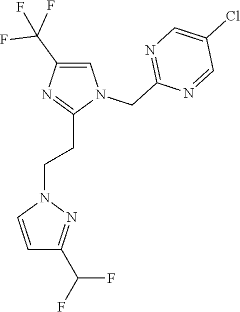 [SMILES: FC(F)c1ccn(CCc2nc(C(F)(F)F)cn2Cc2ncc(Cl)cn2)n1] | 8.63 (s, 2 H), 7.36 (d, 1 H), 7.25 (s, 1 H), 6.65 (t, 1 H), 6.39 (d, 1 H), 5.09 (s, 2 H), 4.66 (t, 2 H), 3.30 (t, 2 H) |
| 1.033 | 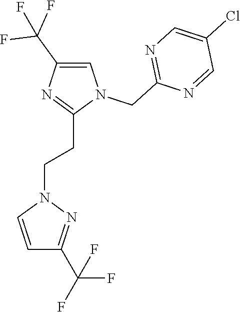 [SMILES: FC(F)(F)c1cn(Cc2ncc(Cl)cn2)c(CCn2ccc(C(F)(F)F)n2)n1] | 8.63 (s, 2 H), 7.43 (d, 1 H), 7.25 (d, 1 H), 6.42 (d, 1 H), 5.14 (s, 2 H), 4.71 (t, 2 H), 3.33 (t, 2 H) |
| 1.034 | 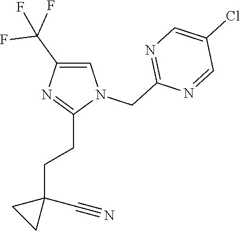 [SMILES: N#CC1(CCc2nc(C(F)(F)F)cn2Cc2ncc(Cl)cn2)CC1] | |
| 1.035 | 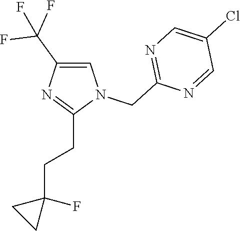 [SMILES: FC1(CCc2nc(C(F)(F)F)cn2Cc2ncc(Cl)cn2)CC1] | |
| 1.036 | 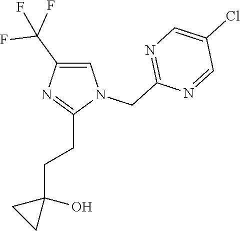 [SMILES: OC1(CCc2nc(C(F)(F)F)cn2Cc2ncc(Cl)cn2)CC1] | |
| 1.037 | 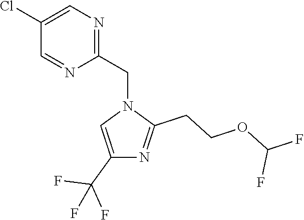 [SMILES: FC(F)OCCc1nc(C(F)(F)F)cn1Cc1ncc(Cl)cn1] | |
| 1.038 | 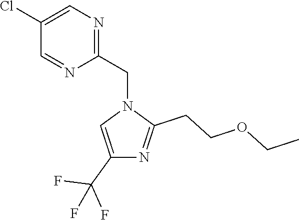 [SMILES: CCOCCc1nc(C(F)(F)F)cn1Cc1ncc(Cl)cn1] | 8.69 (s, 2 H), 7.32 (m, 1 H), 5.42 (s, 2 H), 3.78 (t, 2 H), 3.47 (q, 2 H), 3.05 (t, 2 H), 1.14 (t, 3 H) |
| 1.039 | 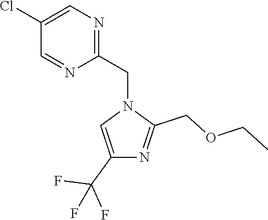 [SMILES: CCOCc1nc(C(F)(F)F)cn1Cc1ncc(Cl)cn1] | 8.68 (s, 2 H), 7.39 (s, 1 H), 5.47 (s, 2 H), 4.65 (s, 2 H), 3.47 (q, 2 H), 1.09 (t, 3 H) |
| 1.040 | 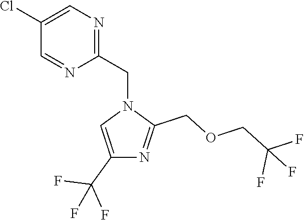 [SMILES: FC(F)(F)COCc1nc(C(F)(F)F)cn1Cc1ncc(Cl)cn1] | |
| 1.041 | 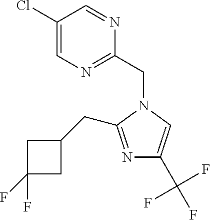 [SMILES: FC1(F)CC(Cc2nc(C(F)(F)F)cn2Cc2ncc(Cl)cn2)C1] | |
| 1.042 | 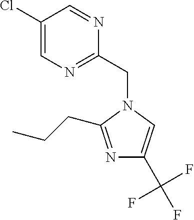 [SMILES: CCCc1nc(C(F)(F)F)cn1Cc1ncc(Cl)cn1] | 8.68 (s, 2 H), 7.30 (q, 1 H), 5.25 (s, 2 H), 2.65 − 2.71 (m, 2 H), 1.75 (dd, 2 H), 0.96 (t, 3 H) |
| 1.043 | 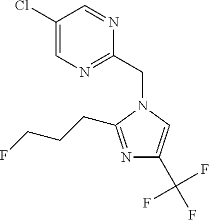 [SMILES: FCCCc1nc(C(F)(F)F)cn1Cc1ncc(Cl)cn1] | 8.69 (s, 2 H), 7.31 (d, 1 H), 5.29 (s, 2 H), 4.58 (t, 1 H), 4.46 (t, 1 H), 2.84-2.90 (m, 2 H), 2.16-2.30 (m, 2 H) |
| 1.044 | 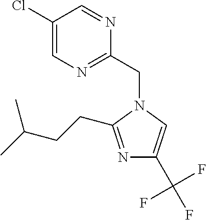 [SMILES: CC(C)CCc1nc(C(F)(F)F)cn1Cc1ncc(Cl)cn1] | |
| 1.045 | 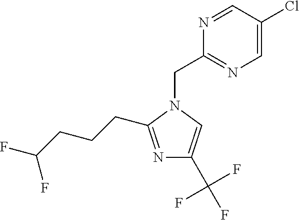 [SMILES: FC(F)CCCc1nc(C(F)(F)F)cn1Cc1ncc(Cl)cn1] | |
| 1.046 | 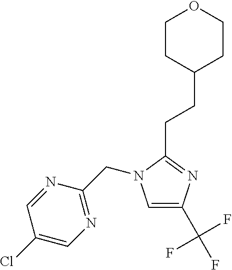 [SMILES: FC(F)(F)c1cn(Cc2ncc(Cl)cn2)c(CCC2CCOCC2)n1] | |
| 1.047 | 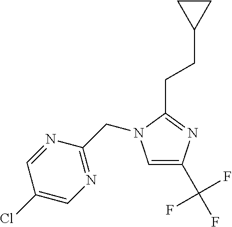 [SMILES: FC(F)(F)c1cn(Cc2ncc(Cl)cn2)c(CCC2CC2)n1] | |
| 1.048 | 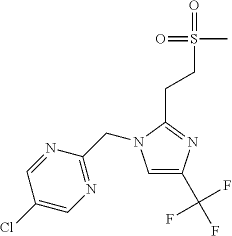 [SMILES: CS(=O)(=O)CCc1nc(C(F)(F)F)cn1Cc1ncc(Cl)cn1] | 1H NMR (400 MHz, ACETONITRILE-d3) δ = 8.66 (s, 2 H), 7.44 (s, 1 H), 5.32 (s, 2 H), 3.40 − 3.47 (m, 2 H), 3.02 − 3.07 (m, 2 H), 2.80 (s, 3 H) |
| 1.049 | 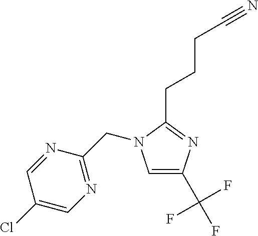 [SMILES: N#CCCCc1nc(C(F)(F)F)cn1Cc1ncc(Cl)cn1] | |
| 1.050 | 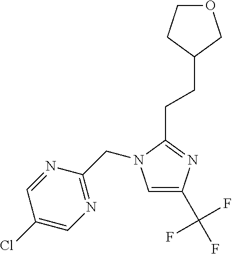 [SMILES: FC(F)(F)c1cn(Cc2ncc(Cl)cn2)c(CCC2CCOC2)n1] | |
| 1.051 | 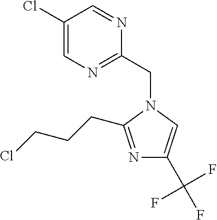 [SMILES: FC(F)(F)c1cn(Cc2ncc(Cl)cn2)c(CCCCl)n1] | 8.68 (s, 2 H), 7.30 (d, 1 H), 5.30 (s, 2 H), 3.63 (t, 2 H), 2.90 (t, 2 H), 2.25 − 2.33 (m, 2 H) |
| 1.052 | 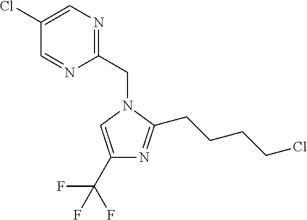 [SMILES: FC(F)(F)c1cn(Cc2ncc(Cl)cn2)c(CCCCCl)n1] | |
| 1.053 | 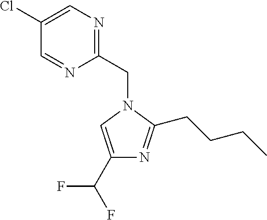 [SMILES: CCCCc1nc(C(F)F)cn1Cc1ncc(Cl)cn1] | 8.68 (s, 2 H), 7.21 (t, 1 H), 6.67 (t, 1 H), 5.26 (s, 2 H), 2.68 −2.74 (m, 2 H), 1.65 − 1.79 (m, 2 H), 1.34 − 1.44 (m, 2 H), 0.92 (t, 3 H) |
| 1.054 | 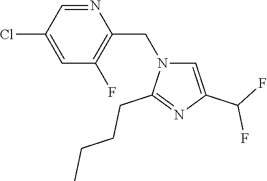 [SMILES: CCCCc1nc(C(F)F)cn1Cc1ncc(Cl)cc1F] | 8.40 (d, 1 H), 7.53 (dd, 1 H), 7.19 (t, 1 H), 6.66 (t, 1 H), 5.21 (d, 2 H), 2.76 − 2.82 (m, 2 H), 1.69 − 1.78 (m, 2 H), 1.42 (m, 2 H), 0.94 (t, 3 H) |
| 1.055 | 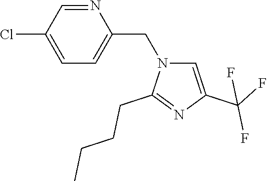 [SMILES: CCCCc1nc(C(F)(F)F)cn1Cc1ccc(Cl)cn1] | 8.48 (d, 1 H), 7.43 (m, 1 H), 7.25 (s, 1 H), 6.98 (m, 1 H), 5.20 (s, 2 H), 2.65 − 2.72 (m, 2 H), 1.61 − 1.80 (m, 2 H), 1.38 (m, 2 H), 0.92 (t, 3 H) |
| 1.056 | 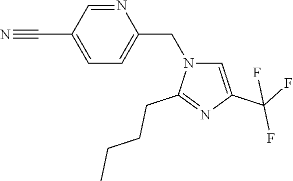 [SMILES: CCCCc1nc(C(F)(F)F)cn1Cc1ccc(C#N)cn1] | 8.89 (d, 1 H), 7.99 (dd, 1 H), 7.25 (d, 1 H), 7.03 (d, 1 H), 5.28 (s, 2 H), 2.60 − 2.67 (m, 2 H), 1.61 − 1.75 (m, 2 H), 1.30 − 1.42 (m, 2 H), 0.90 (t, 3 H) |
| 1.057 | 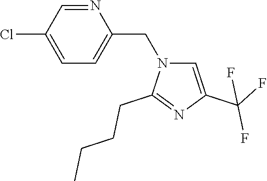 [SMILES: CCCCc1nc(C(F)(F)F)cn1Cc1ccc(Cl)cn1] | 8.56 (d, 1 H), 7.68 (dd, 1 H), 7.23 (s, 1 H), 6.89 (d, 1 H), 5.17 (s, 2 H), 2.62 − 2.69 (m, 2 H), 1.61 − 1.76 (m, 2 H), 1.37 (m, 2 H), 0.90 (t, 3 H) |
| 1.058 | 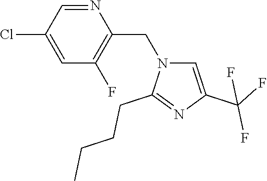 [SMILES: CCCCc1nc(C(F)(F)F)cn1Cc1ncc(Cl)cc1F] | 8.40 (dd, 1 H), 7.53 (dd, 1 H), 7.27 (s, 1 H), 5.21 (d, 2 H), 2.74 − 2.81 (m, 2 H), 1.64 − 1.80 (m, 2 H), 1.34 − 1.49 (m, 2 H), 0.94 (t, 3 H) |
| 1.059 | 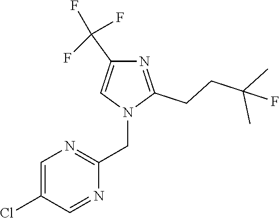 [SMILES: CC(C)(F)CCc1nc(C(F)(F)F)cn1Cc1ncc(Cl)cn1] | |
| 1.060 | 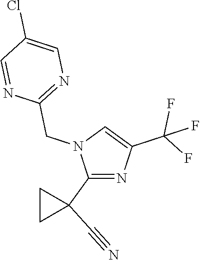 [SMILES: N#CC1(c2nc(C(F)(F)F)cn2Cc2ncc(Cl)cn2)CC1] | ¹H NMR (400 MHz, METHANOL- d4) δ = 8.82 (s, 2 H), 7.77 (s, 1 H), 5.73 (s, 2 H), 1.62 − 1.73 (m, 4 H) |
| 1.061 | 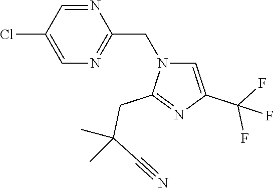 [SMILES: CC(C)(C#N)Cc1nc(C(F)(F)F)cn1Cc1ncc(Cl)cn1] | 8.70 (s, 2 H), 7.41 (s, 1 H), 5.47 (s, 2 H), 3.11 (s, 2 H), 1.54 (s, 6 H) |
| 1.062 | 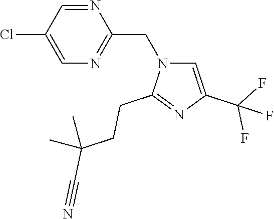 [SMILES: CC(C)(C#N)CCc1nc(C(F)(F)F)cn1Cc1ncc(Cl)cn1] | 8.70 (s, 2 H), 7.27 (s, 1 H), 5.32 (s, 2 H), 2.89-2.93 (m, 2 H), 2.02-2.09 (m, 2 H), 1.41 (s, 6 H) |
| 1.063 | 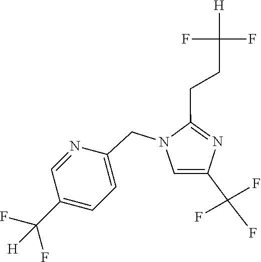 [SMILES: FC(F)CCc1nc(C(F)(F)F)cn1Cc1ccc(C(F)F)cn1] | |
| 1.064 | 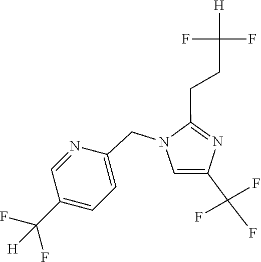 [SMILES: FC(F)CCc1nc(C(F)(F)F)cn1Cc1ccc(C(F)F)cn1] | |
| 1.065 | 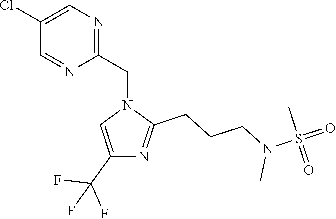 [SMILES: CN(CCCc1nc(C(F)(F)F)cn1Cc1ncc(Cl)cn1)S(C)(=O)=O] | 8.68 (s, 2 H), 7.31 (s, 1 H), 5.29 (s, 2 H), 3.21 (t, 2 H), 2.84 (m, 3 H), 2.77 (s, 3 H), 2.61 (t, 2 H), 2.14 (t, 2 H) |
| 1.066 | 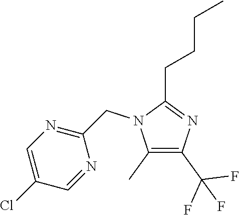 [SMILES: CCCCc1nc(C(F)(F)F)c(C)n1Cc1ncc(Cl)cn1] | |
| 1.067 | 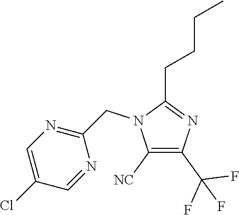 [SMILES: CCCCc1nc(C(F)(F)F)c(C#N)n1Cc1ncc(Cl)cn1] | |
| 1.068 | 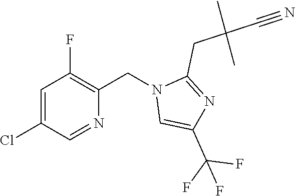 [SMILES: CC(C)(C#N)Cc1nc(C(F)(F)F)cn1Cc1ncc(Cl)cc1F] | 8.41 (dd, 1 H), 7.55 (dd, 1 H), 7.37 (s, 1 H), 5.42 (d, 2 H), 3.17 (s, 2 H) 1.64 (s, 6 H) |
| 1.069 | 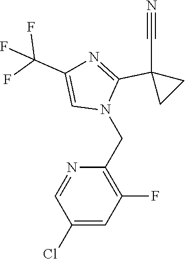 [SMILES: N#CC1(c2nc(C(F)(F)F)cn2Cc2ncc(Cl)cc2F)CC1] | 1H NMR (400 MHz, METHANOL- d4) δ ppm 8.41 (d, 1 H), 7.88 (dd, 1 H), 7.74 (s, 1 H), 5.68 (d, 2 H), 1.60 − 1.76 (m, 4 H) |
| 1.070 | 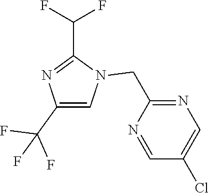 [SMILES: FC(F)c1nc(C(F)(F)F)cn1Cc1ncc(Cl)cn1] | 8.69 (s, 2 H), 7.52 (s, 1 H), 6.78 (t, 1 H), 5.56 (s, 2 H) |
| 1.071 | 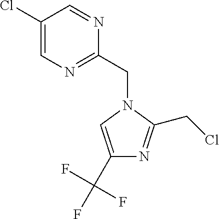 [SMILES: FC(F)(F)c1cn(Cc2ncc(Cl)cn2)c(CCl)n1] | 8.70 (s, 2 H), 7.42 (d, 1 H), 5.46 (s, 2 H), 4.79 (s, 2 H) |
| 1.072 | 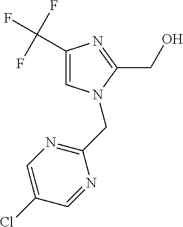 [SMILES: OCc1nc(C(F)(F)F)cn1Cc1ncc(Cl)cn1] | 8.68 (s, 2 H), 7.33 (s, 1 H), 5.47 (s, 2 H), 4.79 (s, 2 H) |
| 1.073 | 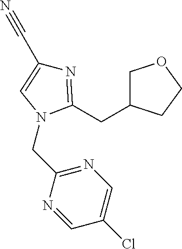 [SMILES: N#Cc1cn(Cc2ncc(Cl)cn2)c(CC2CCOC2)n1] | 8.67 − 8.70 (m, 2 H), 7.54 (s, 1 H), 5.28 (s, 2 H), 3.85 − 3.94 (m, 2 H), 3.76 (m, 1 H), 3.47 (m, 1 H), 2.73 − 2.85 (m, 3 H), 2.15 (m, 1 H), 1.60 − 1.65 (m, 1 H) |
| 1.074 | 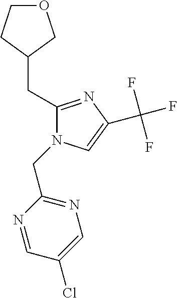 [SMILES: FC(F)(F)c1cn(Cc2ncc(Cl)cn2)c(CC2CCOC2)n1] | 8.69 (s, 2 H), 7.31 (s, 1 H), 5.27 (s, 2 H), 3.86 − 3.93 (m, 2 H), 3.73 − 3.80 (m, 1 H), 3.48 (dd, 1 H), 2.76 − 2.83 (m, 3 H), 2.03 − 2.22 (m, 1 H), 1.65 (m, 1 H) |
| 1.075 | 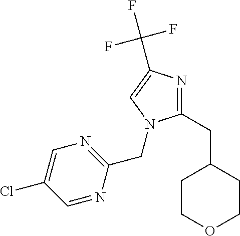 [SMILES: FC(F)(F)c1cn(Cc2ncc(Cl)cn2)c(CC2CCOCC2)n1] | 8.69 (s, 2 H), 7.31 (s, 1 H), 5.27 (s, 2 H), 3.93 (br dd, 2 H), 3.37 (m, 2 H), 2.66 (d, 2 H), 2.11 (m, 1 H), 1.59 − 1.62 (m, 1 H), 1.21 − 1.43 (m, 3 H) |
| 1.076 | 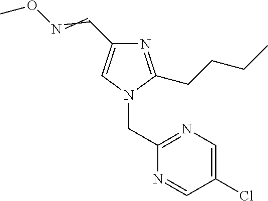 [SMILES: CCCCc1nc(C=NOC)cn1Cc1ncc(Cl)cn1] | 3.3:1 mixture of isomer 1.076-1 and 1.076-2 respectively. 1.076-1: 8.68 (s, 2 H), 7.77 (s, 1 H), 7.54 (s, 1 H), 5.29 (s, 2 H), 4.02 (s, 3 H), 2.70-2.74 (m, 2 H), 1.69 − 1.73 (m, 2 H), 1.35 − 1.41 (m, 2 H), 0.89 − 0.93 (m, 3 H). 1.076-2: 8.67 (s, 2 H), 8.03 (s, 1 H), 7.22 (s, 1 H), 5.65 (s, 2 H), 3.93 (s, 3H), 2.70-2.74 (m, 2 H), 1.69 − 1.73 (m, 2 H), 1.35 − 1.41 (m, 2 H), 0.89 − |
| | | 0.93 (m, 3 H). |
| 1.077 | 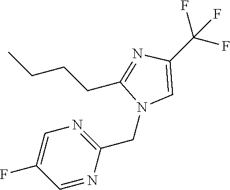 [SMILES: CCCCc1nc(C(F)(F)F)cn1Cc1ncc(F)cn1] | 8.59 (s, 2 H), 7.31 (d, 1 H), 5.28 (s, 2 H), 2.69 − 2.75 (m, 2 H), 1.71- 1.78 (s, 2 H), 1.38 (br d, 2 H), 0.91 (t, 3 H). |
| 1.078 | 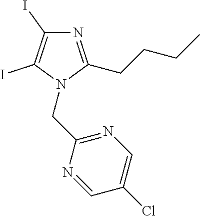 [SMILES: CCCCc1nc(I)c(I)n1Cc1ncc(Cl)cn1] | 8.67 (s, 2 H), 5.38 (s, 2 H), 2.68 − 2.76 (m, 2 H), 1.63-1.69 (m, 2 H), 1.29-1.40 (m, 2 H), 0.88 (t, 3 H) |
| 1.079 | 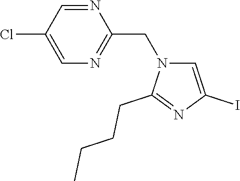 [SMILES: CCCCc1nc(I)cn1Cc1ncc(Cl)cn1] | 8.67 (s, 2 H), 7.03 (s, 1 H), 5.21 (s, 2 H), 2.64 − 2.73 (m, 2 H), 1.63-1.73 (m, 2 H), 1.30-1.43 (m, 2 H), 0.87 − 0.94 (m, 3 H) |
| 1.081 | 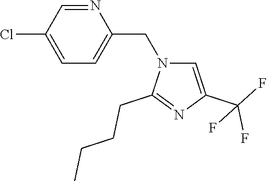 [SMILES: CCCCc1nc(C(F)(F)F)cn1Cc1ccc(Cl)cn1] | 8.66 (d, 1 H), 7.83 (dd, 1 H), 7.23 (d, 1 H), 6.83 (d, 1 H), 5.15 (s, 2 H), 2.62 − 2.68 (m, 2 H), 1.64 − 1.73 (m, 2 H), 1.31 − 1.43 (m, 2 H), 0.90 (t, 3 H) |
| 1.082 | 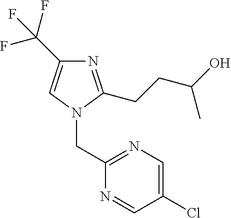 [SMILES: CC(O)CCc1nc(C(F)(F)F)cn1Cc1ncc(Cl)cn1] | 1H NMR (400 MHz, DMSO-d6) δ = 8.95 (s, 2 H), 7.76 (q, 1 H), 5.50 (s, 2 H), 4.47 (d, 1 H), 3.56 (br s, 1 H), 2.53 − 2.68 (m, 2 H), 1.55 − 1.71 (m, 2 H), 1.01 (d, 3 H) |
| 1.083 | 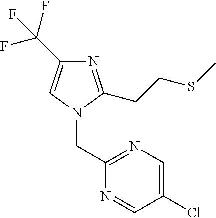 [SMILES: CSCCc1nc(C(F)(F)F)cn1Cc1ncc(Cl)cn1] | 8.69 (s, 2 H), 7.27 (s, 1 H), 5.32 (s, 2 H), 3.00 − 3.06 (m, 2 H), 2.90 − 2.98 (m, 2 H), 2.11 (s, 3 H) |
| 1.084 | 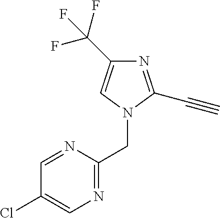 [SMILES: C#Cc1nc(C(F)(F)F)cn1Cc1ncc(Cl)cn1] | 8.70 (s, 2 H), 7.44 (s, 1 H), 5.47 (s, 2 H), 3.31 (s, 1 H) |
| 1.085 | 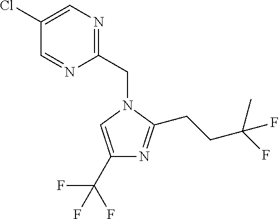 [SMILES: CC(F)(F)CCc1nc(C(F)(F)F)cn1Cc1ncc(Cl)cn1] | 8.69 (s, 2 H), 7.32 (q, 1 H), 5.29 (s, 2 H), 2.90 − 2.97 (m, 2 H), 2.38 − 2.51 (m, 2 H), 1.63 1.70 (t, 3 H). |
| 1.086 | 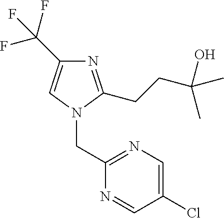 [SMILES: CC(C)(O)CCc1nc(C(F)(F)F)cn1Cc1ncc(Cl)cn1] | 1H NMR (400 MHz, DMSO-d6) δ = 8.96 (s, 2 H), 7.75 (q, 1 H), 5.50 (s, 2 H), 4.24 (s, 1 H), 2.56 − 2.62 (m, 2 H), 1.64 − 1.72 (m, 2 H), 1.04 (s, 6 H). |

### Biological Examples

Seeds of a variety of test species are sown in standard soil in pots Amaranthus pa/meri (AMAPA), Amaranthus retoflexus (AMARE), Echinochloa crus-gali (ECHCG), Ipomoea hederacea (IPOHE), Setaria faberi (SETFA). After cultivation for one day (pre-emergence) or after 8 days cultivation (post-emergence) under controlled conditions in a glasshouse (at 24/16° C., day/night; 14 hours light; 65% humidity), the plants are sprayed with an aqueous spray solution derived from the formulation of the technical active ingredient in acetone/water (50:50) solution containing 0.5% Tween 20 (polyoxyethelyene sorbitan monolaurate, CAS RN 9005-64-5). Compounds are applied at 250 g/ha unless otherwise stated. The test plants are then grown in a glasshouse under controlled conditions in a glasshouse (at 24/16° C., day/night; 14 hours light; 65% humidity) and watered twice daily. After 13 days for pre and post-emergence, the test is evaluated for the percentage damage caused to the plant. The biological activities are shown in the following table on a five-point scale (5=81-100%; 4=61-80%; 3=41-60%; 2=21-40%; 1=0-20%).

TABLE B1
Post-emergence Test

| | Rate | | | | | |
|---|---|---|---|---|---|---|
| Compound | (g/ha) | AMARE | AMAPA | SETFA | ECHCG | IPOHE |
| 1.001 | 250 | 2 | 2 | 1 | 1 | 2 |
| 1.002 | 250 | 5 | 5 | 5 | 5 | 5 |
| 1.003 | 250 | 2 | 2 | 1 | 1 | 2 |
| 1.004 | 250 | 4 | 4 | 4 | 3 | 3 |
| 1.005 | 250 | 3 | 3 | — | 1 | 2 |
| 1.006 | 250 | 3 | 3 | — | 1 | 2 |
| 1.007 | 250 | 4 | 4 | — | 1 | 3 |
| 1.008 | 250 | 1 | 1 | 1 | 1 | 1 |
| 1.009 | 250 | 1 | 1 | — | 1 | 1 |
| 1.010 | 250 | 4 | 4 | — | 4 | 4 |
| 1.011 | 250 | 2 | 2 | 1 | 1 | 3 |
| 1.012 | 250 | 1 | 1 | — | 1 | 2 |
| 1.013 | 250 | 1 | 1 | — | 1 | 1 |
| 1.015 | 250 | 2 | 1 | — | 1 | 3 |
| 1.016 | 250 | 2 | 1 | — | 1 | 2 |
| 1.017 | 250 | 4 | 4 | — | 3 | 4 |
| 1.018 | 250 | 2 | 2 | — | 1 | 3 |
| 1.019 | 250 | 2 | 2 | — | 3 | 1 |
| 1.020 | 250 | 3 | 2 | 1 | 2 | 2 |
| 1.021 | 250 | 3 | 3 | 1 | 1 | 3 |
| 1.022 | 250 | 3 | 3 | 1 | 1 | 3 |
| 1.023 | 250 | 1 | 1 | 1 | 1 | 2 |
| 1.025 | 250 | 5 | 5 | 5 | 5 | 4 |
| 1.026 | 250 | 5 | 5 | 2 | 2 | 4 |
| 1.027 | 250 | 2 | 2 | 1 | 1 | 2 |
| 1.028 | 250 | 5 | 4 | — | 2 | 2 |
| 1.029 | 250 | 4 | 4 | — | 1 | 3 |
| 1.032 | 250 | 5 | 4 | 3 | 3 | 4 |
| 1.033 | 250 | 4 | 3 | 3 | 3 | 4 |
| 1.038 | 250 | 4 | 3 | 3 | 3 | 4 |
| 1.039 | 250 | 2 | 2 | 1 | 1 | 2 |
| 1.042 | 250 | 3 | 3 | — | 1 | 3 |
| 1.048 | 250 | 4 | 4 | 1 | 1 | 4 |
| 1.051 | 250 | 5 | 4 | — | 4 | 4 |
| 1.053 | 250 | 1 | 1 | — | 1 | 1 |
| 1.054 | 250 | 3 | 2 | 1 | 1 | 2 |
| 1.055 | 250 | 1 | 1 | — | 1 | 1 |
| 1.056 | 250 | 2 | 2 | — | 1 | 2 |
| 1.057 | 250 | 3 | 2 | — | 1 | 2 |
| 1.058 | 250 | 4 | 3 | — | 4 | 4 |
| 1.060 | 250 | 2 | 2 | 1 | 1 | 2 |
| 1.061 | 250 | 4 | 3 | 1 | 1 | 4 |
| 1.062 | 250 | 2 | 2 | 2 | 1 | 3 |
| 1.069 | 250 | 4 | 4 | 1 | 1 | 4 |
| 1.070 | 250 | 1 | 1 | 1 | 1 | 2 |
| 1.071 | 250 | 1 | 1 | 1 | 1 | 1 |
| 1.073 | 250 | 2 | 2 | 1 | 1 | 3 |
| 1.074 | 250 | 4 | 4 | — | 3 | 4 |
| 1.075 | 250 | 4 | 4 | — | 4 | 4 |
| 1.076 | 250 | 1 | 1 | — | 1 | 1 |
| 1.077 | 250 | 3 | 2 | — | 1 | 3 |
| 1.078 | 250 | 2 | 2 | — | 1 | 1 |
| 1.079 | 250 | 1 | 1 | — | 1 | 1 |
| 1.081 | 250 | 3 | 3 | — | 1 | 2 |
| 1.082 | 250 | 2 | 3 | 1 | 1 | 1 |
| 1.083 | 250 | 4 | 3 | 1 | 1 | 4 |
| 1.084 | 250 | 2 | 2 | 1 | 1 | 1 |
| 1.085 | 250 | 5 | 5 | 5 | 4 | 4 |
| 1.086 | 250 | 2 | 2 | 1 | 1 | 3 |

TABLE B2
Pre-emergence Test

| | Rate | | | | | |
|---|---|---|---|---|---|---|
| Compound | (g/ha) | AMARE | AMAPA | SETFA | ECHCG | IPOHE |
| 1.001 | 250 | 1 | 2 | 1 | 1 | 1 |
| 1.002 | 250 | 5 | 5 | 5 | 5 | 5 |
| 1.003 | 250 | 1 | 1 | 1 | 1 | 2 |
| 1.004 | 250 | 5 | 5 | 4 | 3 | 2 |
| 1.005 | 250 | 1 | 1 | — | 1 | 1 |
| 1.006 | 250 | 5 | 2 | — | 1 | 1 |
| 1.007 | 250 | 5 | 5 | — | 2 | 5 |
| 1.008 | 250 | 1 | 1 | 1 | 2 | 1 |
| 1.009 | 250 | 1 | 1 | — | 1 | 2 |
| 1.010 | 250 | 5 | 5 | — | 4 | 5 |
| 1.011 | 250 | 1 | 1 | 1 | 1 | 1 |
| 1.012 | 250 | 1 | 1 | — | 1 | 1 |
| 1.013 | 250 | 5 | 2 | — | 1 | 1 |
| 1.015 | 250 | 4 | 4 | — | 1 | 2 |
| 1.016 | 250 | 5 | 5 | — | 1 | 2 |
| 1.017 | 250 | 5 | 5 | — | 5 | 5 |
| 1.018 | 250 | 5 | 4 | — | 1 | 4 |
| 1.019 | 250 | 5 | 5 | — | 5 | 1 |
| 1.020 | 250 | 5 | 5 | 4 | 5 | 4 |
| 1.021 | 250 | 3 | 5 | 1 | 1 | 4 |
| 1.022 | 250 | 3 | 2 | 1 | 1 | 4 |
| 1.023 | 250 | 2 | 2 | 1 | 1 | 1 |
| 1.025 | 250 | 5 | 5 | 5 | 5 | 5 |
| 1.026 | 250 | 5 | 5 | 5 | 3 | 3 |
| 1.027 | 250 | 4 | 1 | 1 | 1 | 1 |
| 1.028 | 250 | 5 | 5 | — | 1 | 4 |
| 1.029 | 250 | 5 | 5 | — | 2 | 5 |
| 1.032 | 250 | 5 | 5 | 5 | 5 | 5 |
| 1.033 | 250 | 5 | 5 | 4 | 3 | 5 |
| 1.038 | 250 | 5 | 5 | 5 | 5 | 5 |
| 1.039 | 250 | 4 | 3 | 2 | 1 | 2 |
| 1.042 | 250 | 4 | 4 | — | 1 | 4 |
| 1.048 | 250 | 5 | 4 | 1 | 1 | 5 |
| 1.051 | 250 | 5 | 5 | — | 2 | 5 |
| 1.053 | 250 | 1 | 1 | — | 1 | 1 |
| 1.054 | 250 | 3 | 4 | 1 | 1 | 1 |
| 1.055 | 250 | 1 | 1 | — | 1 | 3 |
| 1.056 | 250 | 4 | 4 | — | 1 | 2 |
| 1.057 | 250 | 3 | 5 | — | 1 | 1 |
| 1.058 | 250 | 5 | 5 | — | 5 | 5 |
| 1.060 | 250 | 2 | 2 | 1 | 1 | 3 |
| 1.061 | 250 | 3 | 3 | 1 | 1 | 4 |
| 1.062 | 250 | 5 | 5 | 4 | 4 | 5 |
| 1.069 | 250 | 5 | 4 | 1 | 1 | 4 |
| 1.070 | 250 | 5 | 5 | 1 | 1 | 3 |
| 1.071 | 250 | 3 | 2 | 1 | 1 | 1 |
| 1.073 | 250 | 2 | 2 | 1 | 1 | 2 |
| 1.074 | 250 | 5 | 5 | — | 3 | 5 |
| 1.075 | 250 | 5 | 5 | — | 4 | 5 |
| 1.076 | 250 | 1 | 1 | — | 1 | 1 |
| 1.077 | 250 | 5 | 5 | — | 1 | 5 |
| 1.078 | 250 | 1 | 1 | — | 1 | 1 |
| 1.079 | 250 | 1 | 1 | — | 1 | — |
| 1.081 | 250 | 2 | 2 | — | 1 | 1 |
| 1.082 | 250 | 4 | 2 | 2 | 1 | 1 |
| 1.083 | 250 | 4 | 3 | 1 | 1 | 5 |
| 1.084 | 250 | 2 | 1 | 1 | 1 | 1 |
| 1.085 | 250 | 5 | 5 | 5 | 5 | 5 |
| 1.086 | 250 | 1 | 1 | 1 | 1 | 3 |

1. A compound of Formula (I): 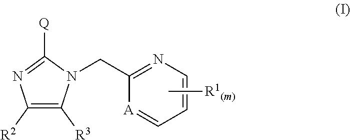
or an agronomically acceptable salt thereof,
wherein
A is CR¹¹ or N;
Q is selected from the group consisting of C₂-C₈ alkyl, C₂-C₈ alkenyl, C₂-C₈ alkynyl, C₁-C₈ haloalkyl, C₂-C₈ haloalkenyl, C₂-C₈ haloalkynyl, C₁-C₄alkoxy-C₁-C₈alkyl-, C₁-C₄haloalkoxy-C₁-C₈alkyl-, C₁-C₄alkoxy-C₁-C₈haloalkyl-, C₁-C₈cyanoalkyl-, C₃-C₆ cycloalkyl (optionally substituted by CN, fluoro or chloro), —S(O)pR⁴ and —(CH₂)nR¹⁰;
R¹ is independently selected from the group consisting of halogen, —CN, C₁-C₂alkyl, C₁-C₂haloalkyl, C₃-C₆cycloalkyl, C₁-C₂alkoxy- and C₁-C₂haloalkoxy-;
R² is selected from the group consisting of halogen, —CN, NO₂, C₁-C₄alkyl, C₁-C₄haloalkyl, C₁-C₄alkoxy, —C(O)C₁-C₄alkyl, —C(O)OC₁-C₄alkyl, C₁-C₄haloalkoxy, —S(O)pC₁-C₄alkyl, —C(R⁶)═NOR⁷ and C₃-C₆cycloalkyl;
R³ is selected from the group consisting of hydrogen, halogen, C₁-C₄ alkyl, C₁-C₄ haloalkyl, C₁-C₄ alkoxy, C₁-C₄ haloalkoxy, —CN, NO₂, C₂-C₄alkenyl, C₂-C₄alkynyl, —S(O)ₚC₁-C₄alkyl, —S(O)ₚC₁-C₄haloalkyl, —C(O)OC₁-C₄alkyl and —C(O)NR⁸R⁹;
R⁴ is selected from the group consisting of C₁-C₄alkyl, C₁-C₄haloalkyl and C₃-C₆cycloalkyl;
R⁵ is selected from the group consisting of hydrogen, methyl, ethyl, —(CH₂)-cyclopropyl, C₂-C₃ alkenyl, C₂-C₃ alkynyl and cyclopropyl;
R⁶ is selected from the group consisting of hydrogen, methyl and ethyl;
R⁷ is methyl or ethyl;
R⁸ is hydrogen or C₁-C₄alkyl;
R⁹ is hydrogen or C₁-C₄alkyl;
R¹⁰ is selected from the group consisting of —OH, —C(H)O, —C(O)R⁴, —S(O)ₚR⁴, —C(H)(OH)—CH₂OH, —C(R⁶)═NOR⁵, —NR¹²R¹³, C₃-C₆ cycloalkyl (optionally substituted by CN, fluoro or hydroxy), a C₃-C₆ saturated heterocycle comprising one or more oxygen atoms, and a 5-membered heteroaryl which comprises from 1 to 3 heteroatoms each independently selected from the group consisting of oxygen, nitrogen and sulphur, wherein said heteroaryl is optionally substituted by one or two substituents independently selected from the group consisting of halogen, cyano, C₁-C₂ alkyl, C₁-C₂ haloalkyl, C₃-C₄cycloalkyl, C₁-C₂ alkoxy and C₁-C₄ haloalkoxy;
R¹¹ is selected from the group consisting of hydrogen, fluoro, chloro and CN;
R¹² and R¹³ are independently selected from the group consisting of C₁-C₄alkyl, C₃-C₄cycloalkyl, C₁-C₄alkyl-S(O)ₚ—, C₃-C₄cycloalkyl-S(O)ₚ—, C₁-C₄alkyl-C(O)— and C₃-C₄cycloalkyl-C(O)—;
m=1 or 2;
n=1,2 or 3; and
p=0, 1 or 2.

2. A compound of Formula (I) according to claim 1, which is of Formula (Ia) 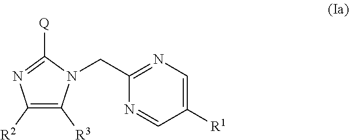
wherein Q, R¹, R² and R³ are as defined in claim 1 above.

3. A compound according to claim 1, wherein R¹ is chloro.

4. A compound according to claim 1, wherein R² is halogen or C₁-C₄haloalkyl.

5. A compound according to claim 1, wherein R³ is hydrogen or halogen.

6. A compound according to claim 1, wherein Q is selected from the group consisting of C₂-C₈ alkyl, C₂-C₈ alkenyl, C₁-C₈ haloalkyl, C₁-C₄alkoxy-C₁-C₈alkyl-, C₁-C₄haloalkoxy-C₁-C₈alkyl-, —S(O)ₚR⁴ and —(CH₂)ₙR¹⁰.

7. A compound according to claim 6, wherein Q is —(CH₂)ₙR¹⁰, n is 1, 2 or 3 and R¹⁰ is selected from the group consisting of —OH, —C(H)(OH)—CH₂OH and —C(H)═NOR⁵.

8. A compound according to claim 6, wherein Q is C₂-C₈ alkyl.

9. A compound according to claim 6, wherein Q is C₁-C₈ haloalkyl.

10. A compound according to claim 6, wherein Q is C₁-C₄haloalkoxy-C₁-C₃alkyl-.

11. A herbicidal composition comprising a compound according to claim 1 and an agriculturally acceptable formulation adjuvant.

12. A herbicidal composition according to claim 11, further comprising at least one additional pesticide.

13. A herbicidal composition according to claim 12, wherein the additional pesticide is a herbicide or herbicide safener.

14. A method of controlling weeds at a locus comprising application to the locus of a weed controlling amount of a composition according to claim 11.

15. Use of a compound of Formula (I) as defined in claim 1 as a herbicide.

16. A compound of Formula Int-1: 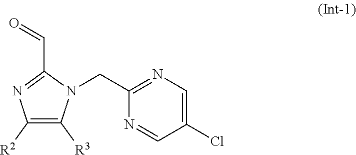
wherein R² and R³ are as defined in claim 1.

17. A compound of Formula (IIIa-1): 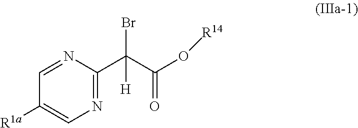
wherein R^{1a} is halogen and R¹⁴ is C₁-C₄ alkyl.
